(19) United States
(12) Patent Application Publication
(10) Pub. No.: US 2023/0158173 A1
(43) Pub. Date: May 25, 2023

(54) GENE THERAPY FOR COCKAYNE SYNDROME

(71) Applicant: UNIVERSITY OF FLORIDA RESEARCH FOUNDATION, INC., Gainesville, FL (US)

(72) Inventors: Christina A. Pacak, Gainesville, FL (US); Peter B. Kang, Gainesville, FL (US)

(21) Appl. No.: 17/912,690

(22) PCT Filed: Mar. 19, 2021
(86) PCT No.: PCT/US2021/023254

§ 371 Date: Sep. 19, 2022

Related U.S. Application Data
(60) Provisional application No. 62/992,729, filed on Mar. 20, 2020.

(51) Int. Cl. A61K 48/00; A61P 25/00; C12N 9/16

(52) U.S. Cl. CPC A61K 48/0066; A61P 25/00; C12N 9/16

(57) ABSTRACT

Provided herein are methods of treating a subject with Cockayne Syndrome (CS) or a predisposition thereto. Also provided are methods of treating a subject with one or more mutations in an ERCC5 gene, an ERCC8 gene, an ERCC6 gene, or a combination thereof, methods of delaying the onset of Cockayne Syndrome (CS), or a symptom thereof, in a subject with one or more mutations in an ERCC5 gene, an ERCC8 gene, an ERCC6 gene, or a combination thereof, and methods of slowing, halting or reversing progression of Cockayne Syndrome (CS) in a subject. In exemplary embodiments, the method comprises administering to the subject a replication-incompetent Adeno-associated Vims (riAAV) comprising a nucleotide sequence encoding a Xeroderma Pigmentosum group G (XPG) protein, a Cockayne Syndrome type A (CSA) protein, or a Cockayne Syndrome type B (CSB) protein in an effective amount. Provided herein are related Adeno-associated Virus and cells comprising the same.

### CROSS-REFERENCE TO RELATED APPLICATION

This application claims the benefit of priority to U.S. Provisional Patent Application No. 62/992,729, filed on Mar. 20, 2020, the disclosure of which is hereby incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 58,056 byte ASCII (Text) file named “54026_Seqlisting.txt”; created on Mar. 18, 2021.

### BACKGROUND

Cockayne syndrome (CS) is a rare disease characterized by neurodegeneration and premature aging throughout the body. CS is caused by mutations in various genes involved in DNA repair mechanisms including excision repair cross-complementing (ERCC) genes. The two most common types are Cockayne Syndrome B (CSB) encoded by the excision repair cross-complementation 6 (ERCC6) gene and Cockayne Syndrome A (CSA) encoded by the ERCC8 gene. The rarest version of CS in humans is caused by mutations in Xeroderma Pigmentosum group G (XPG) protein.

Characterized by abnormal and slow growth and development, CSA becomes evident within the first few years after birth. The outward appearance of afflicted individuals may be described as ‘Cachectic dwarfism’. Other features of CSA include cutaneous photosensitivity, thin, dry hair, a progeroid appearance, progressive pigmentary retinopathy, sensorineural hearing loss, and dental caries. CSA patients often exhibit disproportionately long limbs with large hands and feet, and flexion contractures of joints are usual skeletal features. A ‘horse-riding stance’ is often the result of knee contractures. In CSA, there is delayed neural development and severe progressive neurologic degeneration resulting in mental retardation. The mean age at death in reported cases is 12.5 years, although a few affected individuals have lived into their late teens or twenties. Remarkably, in striking contrast with xeroderma pigmentosum (XP), patients with CSA have no significant increase in skin cancer or infection (Nance and Berry, 1992, Am J Med Genet 42:68-84).

Approximately 80% of CSA patients have additional mutations in the ERCC6 gene (Licht et al., J Hum Genet 73:1217-1239 (2003)). The congenital severe phenotype includes severe failure to thrive, severe mental retardation, congenital cataracts, loss of adipose tissue, joint contractures, distinctive face with small, deep-set eyes and prominent nasal bridge, kyphosis, and cachectic dwarfism. Patients with the ERCC6 gene mutations can exhibit sensorineural hearing loss, no language skills, inability to sit or walk independently, and the patients can die by the age of 5 years.

Mutations in ERCC5 can lead to xeroderma pigmentosum (XP) and (CS) cockayne syndrome. Patients with XP/CS or XPG may exhibit extreme microcephaly, dysmorphism, and sun-sensitive skin with several pigmented spots. In other patients, the disease manifested as psychomotor retardation, microcephaly, and was severely sunlight-sensitive with several pigmented cutaneous spots.

Currently, CS treatment is limited to supportive care only and may include educational programs for developmental delay, physical therapy, gastrostomy tube placement as needed. The only options for medications are those for treating spasticity and tremors. Sunscreens and sunglasses and the treatment for hearing loss and cataracts are provided on an as-needed basis. Therefore, there is a great need for effective treatments for CS.

### SUMMARY

Provided herein for the first time are data supporting gene therapy for Cockayne Syndrome (CS), wherein administration of an AAV comprising a codon-optimized sequence for the expression of XPG in subject exhibiting a CS phenyotype led to increased survival, good biodistribution, and a delayed onset of neurodegeneration. Accordingly, the present disclosure provides methods of treating a subject with Cockayne Syndrome (CS) or a predisposition thereto. In exemplary embodiments, the method comprises administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to treat CS in the subject.

Methods of treating a subject with one or more mutations in an ERCC5 gene, an ERCC8 gene, an ERCC6 gene, or a combination thereof are also provided. In exemplary embodiments, the method comprises administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to treat CS in the subject.

Additionally provided by the present disclosure are methods of delaying the onset of Cockayne Syndrome (CS), or a symptom thereof, in a subject with one or more mutations in an ERCC5 gene, an ERCC8 gene, an ERCC6 gene, or a combination thereof. In exemplary embodiments, the method comprises administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to delay the onset of CS or the symptom thereof in the subject.

The present disclosure also provides methods of slowing, halting or reversing progression of Cockayne Syndrome (CS) in a subject. In exemplary embodiments, the methods comprise administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to slow, halt or reverse the progression in the subject.

Further provided are replication-incompetent Adeno-associated Virus serotype 9 (riAAV9). In exemplary embodiments, the riAAV9 comprises a human codon optimized ERCC8 gene, optionally, SEQ ID NO: 8, and comprising a promoter of SEQ ID NO: 7, a mutated double-stranded ITR of SEQ ID NO: 6 and an ITR of SEQ ID NO: 9). In exemplary embodiments, the riAAV9 comprises a human codon optimized ERCC6 gene, optionally, SEQ ID NO: 14 a truncated poly A sequence comprising a sequence of SEQ ID NO: 4 and a sequence of SEQ ID NO: 5, a ubiquitous promoter up to 200 nt long, an ITR of SEQ ID NO:10 and an ITR of SEQ ID NO: 13, wherein the AAV is a single-stranded AAV plasmid. In exemplary embodiments, the riAAV9 comprises a human codon optimized ERCC5 gene, optionally, SEQ ID NO: 12, a promoter comprising SEQ ID NO: 11, an ITR of SEQ ID NO:10 and an ITR of SEQ ID NO: 13.

The disclosure further provides use of the AAV described herein (e.g., a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein, or a combination thereof) in the treatment of a subject with Cockayne Syndrome (CS) or a predisposition thereto; in the treatment of a subject with one or more mutations in an ERCC5 gene, an ERCC8 gene, an ERCC6 gene, or a combination thereof; and for delaying the onset of Cockayne Syndrome (CS), or a symptom thereof, in a subject with one or more mutations in an ERCC5 gene, an ERCC8 gene, an ERCC6 gene, or a combination thereof. The disclosure also provides use of the AAV described herein (e.g., a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein, or a combination thereof) in the preparation of a medicament for treating a subject with Cockayne Syndrome (CS) or a predisposition thereto; treating a subject with one or more mutations in an ERCC5 gene, an ERCC8 gene, an ERCC6 gene, or a combination thereof; and for delaying the onset of Cockayne Syndrome (CS), or a symptom thereof, in a subject with one or more mutations in an ERCC5 gene, an ERCC8 gene, an ERCC6 gene, or a combination thereof.

The present disclosure furthermore provides a human cell comprising the AAV of the present disclosures. Also, uses of the human cells and AAV are provided herein. In exemplary embodiments, the present disclosure provides use of the human cell of the present disclosure for treating a subject with Cockayne Syndrome (CS) or a predisposition thereto, or delaying the onset of Cockayne Syndrome (CS), or symptoms thereof, in a subject with one or more mutations in an ERCC gene. Also, use of the human cell of the present disclosure for slowing, halting or reversing progression of Cockayne Syndrome (CS) in a subject is provided.

### BRIEF DESCRIPTION OF THE DRAWINGS

FIG. 1 is an illustration of the study described in Example 3.

FIG. 2A is a graph of the % of survival of mice of each cohort plotted as a function of time.

FIG. 2B is a graph of the weight (g) of mice of each cohort plotted as a function of time. FIG. 2C is graph of the weight (g) of mice of each cohort as measured at Week 18 of the study.

FIG. 3A is a graph of the length (cm) of mice of each cohort plotted as a function of time.

FIG. 3B is graph of the length (cm) of mice of each cohort as measured at Week 18 of the study.

FIG. 4A is a graph of the % of mice of each cohort exhibiting kyphosis plotted as a function of time. FIG. 4B is graph of % of mice of each cohort exhibiting tremors plotted as a function of time.

FIG. 5 is a graph of the % of mice of each cohort exhibiting ataxia plotted as a function of time.

FIGS. 6A-6E are plots of activity of mice of each cohort as measured by the ActiTrack system. FIG. 6A is a plot of the activity of mice in the WT healthy control cohort, FIG. 6B is a plot of the activity of mice in the Xpg^{-/-} diseased, untreated cohort, FIG. 6C is a plot of the activity of the Xpg^{-/-} mice treated with 10¹³ vg/kg AAV9-ERCC5, FIG. 6D is a plot of the activity of the Xpg^{-/-} mice treated with 3×10¹³ vg/kg AAV9-ERCC5, FIG. 6E is a plot of the activity of the Xpg^{-/-} mice treated with 3×10¹⁴ vg/kg AAV9-ERCC5, as measured at Week 12.

FIG. 7A is a graph of the distance (cm) moved by each cohort as measured at Week 12.

FIG. 7B is a graph of the fast movements made by each cohort as measured at Week 12. FIG. 7C is a graph of the slow movements made by each cohort as measured at Week 12.

FIG. 8A is a graph of the resting time of each cohort as measured at Week 12. FIG. 8B is a graph of the number of rearing of each cohort as measured at Week 12.

FIG. 9A is a plot of the activity of the mice in the WT healthy control cohort. FIG. 9B is a plot of the activity of the mice in the Xpg^{-/-} diseased, untreated cohort, FIG. 9C is a plot of the activity of the Xpg^{-/-} mice treated with 3×10¹⁴ vg/kg AAV9-ERCC5, as measured at Week 12.

FIG. 10A is a graph of the distance moved post-exercise of the WT control, untreated Xpg^{-/-}, and highest dose treated cohorts. FIG. 10B is a graph of the fast movements made post-exercise by the WT control, untreated Xpg^{-/-}, and highest dose treated cohorts and FIG. 10C is a graph of the slow movements made post-exercise by the WT control, untreated Xpg^{-/-}, and highest dose treated cohorts.

FIG. 11A is a graph of the resting time post exercise of the WT control, untreated Xpg^{-/-}, and highest dose treated cohort. FIG. 11B is a graph of the number of rearing of the WT control, untreated Xpg^{-/-}, and highest dose treated cohorts.

FIG. 12A is a graph of the final brain weights of each cohort and FIG. 12B is a graph of the final liver weights of each cohort, as measured after tissue harvest. Measurements were taken at various ages: 18-24 weeks for WT and 12-21 weeks for untreated and treated Xpg^{-/-} groups.

FIG. 13A is a graph of the femur weight of each cohort, FIG. 13B is a graph of the femur length, and FIG. 13C is a graph of the femur diameter, as measured after tissue harvest. Measurements were taken at various ages: 18-24 weeks for WT and 12-21 weeks for untreated and treated Xpg^{-/-} groups.

FIG. 14 is a graph of the amount of vector genome present per diploid cell present in the indicated tissue. Three bars are provided for each tissue and correspond to dose: left bar=1×10¹³ vg/kg; center bar=3×10¹³ vg/kg; right bar=3×10¹⁴ vg/kg.

FIG. 15 is a graph of codon optimized ERCC5 mRNA expression levels of the indicated tissues. Three bars are provided for each tissue and correspond to dose: left bar=1×10¹³ vg/kg; center bar=3×10¹³ vg/kg; right bar=3×10¹⁴ vg/kg.

FIG. 16 is a vector map of the AAV9 comprising a codon optimized nucleotide sequence encoding XPG.

FIG. 17 is a vector map of the AAV9 comprising a codon optimized nucleotide sequence encoding CPA.

### DETAILED DESCRIPTION

The present disclosure provides methods of treating a subject with Cockayne Syndrome (CS) or a predisposition thereto. In exemplary embodiments, the method comprises administering to the subject an Adeno-associated Virus (AAV), e.g., a replication-incompetent Adeno-associated Virus (riAAV), comprising a nucleotide sequence encoding a Xeroderma Pigmentosum group G (XPG) protein, a Cockayne Syndrome type A (CSA) protein, or a Cockayne Syndrome type B (CSB) protein in an amount effective to treat CS in the subject.

Also provided by the present disclosure are methods of treating a subject with one or more mutations in an ERCC5 gene, an ERCC8 gene, an ERCC6 gene, or a combination thereof. In exemplary embodiments, the method comprises administering to the subject an AAV (e.g., riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to treat CS in the subject.

Further provided are methods of delaying the onset of Cockayne Syndrome (CS), or a symptom thereof, in a subject with one or more mutations in an ERCC5 gene, an ERCC8 gene, an ERCC6 gene, or a combination thereof. The method in exemplary embodiments comprises administering to the subject an AAV, e.g., riAAV, comprising a nucleotide sequence encoding a Xeroderma Pigmentosum group G (XPG) protein, a Cockayne Syndrome type A (CSA) protein, or a Cockayne Syndrome type B (CSB) protein in an amount effective to delay the onset of CS or the symptom thereof in the subject.

Methods of slowing, halting or reversing progression of Cockayne Syndrome (CS) in a subject are additionally provided herein. In exemplary embodiments, the method comprises administering to the subject an AAV (e.g., riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to slow, halt or reverse the progression in the subject.

The methods of the present disclosure relate to treating CS. The CS in some aspects is CSA. In other instances, the CS is CSB. In alternative instances, the CS is XPG. As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating Cockayne Syndrome of the present disclosure can provide any amount or any level of treatment. Furthermore, the treatment provided by the method of the present disclosure can include treatment of one or more conditions or symptoms or signs of the CS being treated. Also, the treatment provided by the methods of the present disclosure can encompass slowing, halting or reversing the progression of the CS. For example, the methods can treat CS by virtue of increasing survival, delaying the onset of neurodegeneration, delaying the onset of one or more symptoms, e.g., delaying the onset of kyphosis, ataxia, tremors, decreasing the extent of neurodegeneration, decreasing the extent of symptoms (e.g., decreasing the extent of kyphosis, ataxia, tremors), and the like. In various aspects, the methods treat by way of delaying the onset of CS or a symptom thereof by at least 1 day, 2 days, 4 days, 6 days, 8 days, 10 days, 15 days, 30 days, 1 week, 2 weeks, 3 week, 4 week, 5 weeks, one month, two months, 3 months, 4 months, 6 months, 1 year, 2 years, 3 years, 4 years, or more. In various aspects, the methods treat by way increasing the survival of the subject.

Symptoms of CS include any of those described herein or in the art. See, e.g., Zafeiriou et al., Pediatric Research 49: 407-412 (2001); Nouspikel et al., PNAS 94:3116-6121 (1997); Rapin et al., Neurology 55(10): doi://https://doi.org/10.1212.WNL.55.10.1442; Wilson et al., Genet. Med. 18: 483-493, 2016; Ellaway et al., J. Med. Genet. 37: 553-557, 2000, Mohmoud et al., Am, J. Med. Genet. 111: 81-85, 2002, Nancy and Berry, Am. J. Med. Genet. 42: 68-84, 1992, Traboulsi et al., Am. J. Ophthal. (1992), Patton et al., Am. J. Ophthal. (1989), Burrnback et al, Am. J. Ophthal. (1978), Houston et al., Am. J. Med. Genet. 13: 211-223, 1982, and references listed in Table B, all of which are incorporated herein by reference in their entirety and particularly with respect to discussion of CS symptoms. In exemplary aspects, the symptoms of CS is neurodegeneration, tremors, dystonia, ataxia, hearing loss, vision loss, cataracts, cognitive disability, cachexia (failure to thrive), severe growth defects (short stature), microcephaly, kyphosis, skin photosensitivity, liver failure, renal dysfunction, or a combination thereof. In exemplary aspects, the symptom is small in size gestational age, micropthalmia, bilateral congenital cataracts, hearing impairment, progressive somatic and neurodevelopmental arrest, and infantile spasms, massive photosensitivity with erythema and blistering after minimal sun exposure, small skin cancers. In exemplary aspects, the symptom is microcephaly, dysmorphism, and sun-sensitive skin with several pigmented spots. In exemplary aspects, the symptom is psychomotor retardation, microcephaly, and was severely sunlight-sensitive with several pigmented cutaneous spots. In certain aspects, the symptom is dwarfism, microcephaly, severe mental retardation, ‘pepper-and-salt’ chorioretinitis, intracranial calcification, dementia, gait disturbance, incontinence, leukodystrophy with ‘tigroid’ demyelinization, nystagmus, weakness, hearing loss, clinical photosensitivity, tremor, joint contractures, abnormal liver function tests, and abnormal bowel movements, photophobia, dwarfism, mental retardation, cataracts, retinopathy, and optic atrophy

CS may be diagnosed by one or more ways. CS may be diagnosed prenatally by studying RNA synthesis in cultured amniotic cells after irradiation with ultraviolet light. Cultured cells from CS patients are hypersensitive to the lethal effects of UV and some chemical carcinogens. The normal recovery in DNA and RNA synthesis after UV exposure does not occur (Mayne and Lehmann, 1982, Cancer Res. 42: 1473-1478, 1982). A test based on this observation is simple and rapid and its outcome is unambiguous. Alternatively, CS may be diagnosed by genetic evaluation of the ERCC5, ERCC6, and ERCC8 genes, wherein the presence of mutations known to be associated with or causative of CS are determined.

Accordingly, in various instances, the subject being treated in the methods of the present disclosure has one or more mutations in the ERCC5 gene, the ERCC8 gene, and/or the ERCC6 gene. Optionally, the subject is at least heterozygous for at least one causative mutation in at least one of SEQ ID NOs: 1-3. In some aspects, the mutation is one described in Table B.

TABLE B

| Gene | Mutation | Reference Citation |
|---|---|---|
| ERCC5 | 526C→T | Zafeiriou et al., 2001, supra |
| ERCC5 | 984C→T | Nouspikel et al., 1997, supra |
| ERCC5 | 1 bp deletion within an AAA triplet at nucleotides | Nouspikel et al., 1997, supra |
| | 2170-2172, which resulted in a TGA stop codon after | |
| | amino acid 659. | |
| ERCC8 | | Henning et al., Cell |
| | | 82: 555-564, 1995 |
| ERCC8 | G-to-T transversion, resulting in a glu13-to-ter | Cao et al., J. Hum. Genet. |
| | (E13X) substitution, and A205P | 49: 61-63, 2004. |
| ERCC8 | a 479C-T transition, resulting in an ala160-to-val | Ridley et al., J. Hum. Genet. |
| | (A160V) substitution | 50: 151-154, 2005. |
| ERCC8 | 8 mutations described therein | Bertola et al., J. Hum. Genet. |
| | | 51: 701-705, 2006. |
| ERCC8 | Y322X | Khayat et al., Am. J. Med. |
| | | Genet. 152A: 3091-3094, 2010. |
| ERCC6 | 18 mutations; homozygous 1630G-A transition in | Mallery et al., Am. J. Hum. |
| | the ERCC6 gene, resulting in a trp517-to-ter | Genet. 62: 77-85, 1998. |
| | (W517X); homozygous 2282C-T transition in the | |
| | ERCC6 gene, resulting in an arg735-to-ter (R735X) | |
| | substitution. This same truncating mutation was | |
| | found in compound heterozygous state with an | |
| | arg453-to-ter (R453X; a 1-bp deletion (1597delG) | |
| | in the center of a 12-bp inverted repeat, resulting | |
| | in a stop codon at residue 506, and a 3363G-C | |
| | transversion, resulting in a pro1095-to-arg | |
| | (P1095R; 609413.0008) substitution. | |
| ERCC6 | homozygosity for the R735X mutation in the | Colella et al., Hum. Molec. |
| | ERCC6 gene; homozygous for a 1436C-T transition | Genet. 8: 935-941, 1999. |
| | in the ERCC6 gene, resulting in an arg453-to-ter | |
| | (R453X) substitution. heterozygosity for 2 | |
| | mutations in the ERCC6 gene: a 1-bp insertion | |
| | (1051insA) in codon 325, leading to frameshift and | |
| | creation of a premature termination at codon 368; | |
| | and a 4-bp insertion (1053insTGTC) in codon 659, | |
| | causing a frameshift and creation of a premature | |
| | termination at codon 682; 4-bp insertion | |
| | (1053insTGTC) in the ERCC6 gene | |
| ERCC6 | homozygous 1-bp insertion (1034insT) in exon 5 of | Falik-Zaccai et al., Am. J. Med. |
| | the ERCC6 | Genet. 146A: 1423-1429, 2008 |

In various aspects, the method of the disclosure optionally comprises detecting a mutation in any of the genes described herein. Methods of detecting such mutations are known in the art and include, for example, sequencing a subject's DNA or RNA obtained from a biological sample.

The ERCC5, ERCC6, and ERCC8 genes and sequences encoded by such genes are known in the art and available at the National Center for Biotechnology Information (NCBI) website. Table A below provides a list of sequences relating to the genes and proteins to the SEQ ID NOs: of the sequence listing incorporated herein.

TABLE A

| | | | | Human |
|---|---|---|---|---|
| Cockayne | | Human Amino | Gene Name | Nucleotide |
| Syndrome | Protein | Acid Sequence | (NCBI Gene ID | Sequence |
| Type | Name | (SEQ ID NO:) | for Human) | (SEQ ID NO:) |
| XPG | XPG | 15 | ERCC5 | 3 |
| | | | (2073) | |
| CSA | CSA | 16 | ERCC8 | 1 |
| | | | (1161) | |
| CSB | CSB | 17 | ERCC6 | 2 |
| | | | (2074) | |

AAV

The methods of the present disclosure relate to administering an Adeno-associated virus (AAV) comprising a nucleotide sequence encoding an XPG protein, CSA protein or a CSB protein. In exemplary aspects, the XPG protein is the protein encoded by the human ERCC5 gene. In exemplary aspects, the CSA protein is the protein encoded by the human ERCC8 gene. In exemplary aspects, the CSB protein is the protein encoded by the human ERCC6 gene. In exemplary aspects, the nucleotide sequence is a codon optimized sequence for optimized expression in human cells. In exemplary aspects, the AAV comprises a codon optimized nucleotide sequence encoding the XPG protein, the CSA protein or the CSB protein. For example, the AAV comprises a codon optimized nucleotide sequence encoding the XPG protein comprising the sequence of SEQ ID NO: 12. In exemplary instances, the AAV comprises a codon optimized nucleotide sequence encoding the CSA protein comprising the sequence of SEQ ID NO: 8. In exemplary instances, the AAV comprises a codon optimized nucleotide sequence encoding the CSB protein comprising the sequence of SEQ ID NO: 14.

AAV is a DNA virus not known to cause human disease, making it a desirable gene therapy options. The AAV genome is comprised of two genes, rep and cap, flanked by inverted terminal repeats (ITRs), which contain recognition signals for DNA replication and viral packaging. AAV requires co-infection with a helper virus (i.e., an adenovirus or a herpes virus), or expression of helper genes, for efficient replication. AAV vectors used for administration of a therapeutic nucleic acid typically have a majority of the parental genome deleted, such that only the ITRs remain, although this is not required. Delivering the AAV rep protein enables integration of the AAV vector comprising AAV ITRs into a specific region of genome, if desired. Host cells comprising an integrated AAV genome show no change in cell growth or morphology. As such, prolonged expression of therapeutic factors from AAV vectors can be useful in treating persistent and chronic diseases. In exemplary aspects, the AAV for use in the methods of the present disclosure is based on AAV serotype 9. In alternative aspects, the AAV for use in the method of the present disclosure is based on another AAV, e.g., AAV type 1, AAV type 2, AAV type 3 (including types 3A and 3B), AAV type 4, AAV type 5, AAV type 6, AAV type 7, AAV type 8, AAV type 10, or AAV type 11. The genomic sequences of AAV, as well as the sequences of the ITRs, Rep proteins, and capsid subunits are known in the art. See, e.g., International Patent Publications Nos. WO 00/28061, WO 99/61601, WO 98/11244; as well as U.S. Pat. No. 6,156,303, Srivistava et al. (1983) J Virol. 45:555; Chiorini et al (1998) J Virol. 71:6823; Xiao et al (1999) J Virol. 73:3994; Shade et al (1986) J Virol. 58:921; and Gao et al (2002) Proc. Nat. Acad. Sci. USA 99:11854.

In various aspects, the AAV comprises a viral genome lacking all or part of the native AAV genome. For example, the AAV genome lacks all native AAV protein coding sequences, but retains the AAV ITRs (e.g., AAV9 ITRs), and further comprises the nucleic acid sequence encoding XPG, CSA, or CSB. In exemplary instances, the AAV used in the methods of the present disclosure lack Rep and Cap, and are therefore replication-incompetent (i.e., a replication-incompetent AAV). In various aspects, the AAV comprises a promoter, a sequence encoding the protein of interest (e.g., XPG, CSA, CSB), and a polyA sequence, all of which is flanked by ITRs. In exemplary aspects, when administered to a subject and upon entry into a cell, the AAV persists as an episome within the nucleus of the cell. In some aspects, the AAV rarely integrates into the genome of the cell.

The AAV comprising the nucleotide sequence encoding XPG, CSA, or CSB and further comprising AAV9-based ITRs can be incorporated into an virion (i.e., packaged into a viral capsid) to facilitate introduction of the genome into a cell. AAV capsid proteins compose the exterior, non-nucleic acid portion of the virion and are encoded by the AAV cap gene. The cap gene encodes three viral coat proteins, VP1, VP2 and VP3, which are required for virion assembly. The construction of AAV virions is described in, e.g., U.S. Pat. Nos. 5,173,414; 5,139,941; 5,863,541; 5,869,305; 6,057,152; and 6,376,237; Rabinowitz et al., J. Virol. 76:791-801, 2002; and Bowles et al., J. Virol. 77:423-432, 2003.

Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001, all of which are hereby incorporated by reference, particularly with respect to the discussion of AAV production. Methods for using AAV vectors also are discussed, for example, in Tal, J., J. Biomed. Sci. 7:279-291, 2000 and Monahan and Samulski, Gene delivery 7:24-30, 2000. In some aspects, the AAV is single stranded. In alternative aspects, the AAV is double stranded.

AAV typically contain a variety of nucleic acid sequences necessary for the transcription and translation of an operably linked coding sequence. For example, an expression vector can comprise origins of replication, polyadenylation signals, internal ribosome entry sites (IRES), promoters, enhancers, and the like. The AAV vector of the disclosure preferably comprises a promoter operably linked to the protein coding sequence. “Operably linked” means that a control sequence, such as a promoter, is in a correct location and orientation in relation to another nucleic acid sequence to exert its effect (e.g., initiation of transcription) on the nucleic acid sequence. A promoter can be native or non-native to the nucleic acid sequence to which it is operably linked and native or non-native to a particular target cell type, and the promoter may be, in various aspects, a constitutive promoter, a tissue-specific promoter, or an inducible promoter. Examples of constitutive promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Examples of constitutive mammalian promoters include various housekeeping gene promoters, as exemplified by the β-actin promoter. Inducible promoters and/or regulatory elements are also contemplated for use in the methods described herein. Examples of inducible promoters include, but are not limited to, those from genes such as cytochrome P450 genes, heat shock protein genes, metallothionein genes, and hormone-inducible genes, such as the estrogen gene promoter. Another example of an inducible promoter is the tet promoter that is responsive to tetracycline. Tissue-specific promoters and/or regulatory elements are useful in certain embodiments of the methods described herein. Examples of such promoters include, but are not limited to, the Tie-2 or KDR promoter. In exemplary instances, the promoter is a constitutive promoter. In various aspects, the promoter is a CMV promoter. In exemplary aspects, the CMV promoter comprises the sequence of any one of SEQ ID NO: 7 or 11. In exemplary aspects, the AAV comprises a pair of ITRs. In some aspects, the ITRs are identical and in other aspects, each ITR of the pair are different from one another. In exemplary instances, one of the ITRs is a double stranded mutated ITR. Optionally, such ITR comprises the sequence of SEQ ID NO: 6. In other aspects, the ITR comprises the sequence of SEQ ID NO: 9, 10, or 13. In some aspects, the AAV comprises an ITR of SEQ ID NO: 6 and an ITR of SEQ ID NO: 9. In other aspects, the AAV comprises an ITR of SEQ ID NO: 10 and an ITR of SEQ ID NO: 13. In various instances, the AAV comprises a polyA sequence of SEQ ID NO: 4, optionally, with sequence that is downstream the stop codon of the gene. In some aspects, the polyA sequence comprises SEQ ID NO: 5.

In exemplary aspects, the AAV is a replication-incompetent Adeno-associated Virus serotype 9 (riAAV9) comprising a human codon optimized ERCC8 gene, optionally, SEQ ID NO: 8, and comprising a promoter of SEQ ID NO: 7, a mutated double-stranded ITR of SEQ ID NO: 6 and an ITR of SEQ ID NO: 9.

In exemplary aspects, the AAV is a replication-incompetent Adeno-associated Virus serotype 9 (riAAV9) comprising a human codon optimized ERCC6 gene, optionally, SEQ ID NO: 14 a truncated poly A sequence comprising a sequence of SEQ ID NO: 4 and a sequence of SEQ ID NO: 5, a ubiquitous promoter up to 200 nt long, an ITR of SEQ ID NO:10 and an ITR of SEQ ID NO: 13, wherein the AAV is a single-stranded AAV plasmid.

In exemplary aspects, the AAV is a replication-incompetent Adeno-associated Virus serotype 9 (riAAV9) comprising a human codon optimized ERCC5 gene, optionally, SEQ ID NO: 12, a promoter comprising SEQ ID NO: 11, an ITR of SEQ ID NO:10 and an ITR of SEQ ID NO: 13.

The present disclosure also provides any of the AAV described herein.

With regard to the presently disclosed methods, in various instances, the subject has CS type A (CSA), or a predisposition thereto. In optional aspects, the riAAV comprises a nucleotide sequence encoding a CSA protein, optionally, wherein the nucleotide sequence is a human codon optimized sequence of SEQ ID NO: 1 or comprises the sequence of SEQ ID NO: 8. In certain aspects, the riAAV is a double-stranded AAV plasmid comprising an SV40 poly A sequence and a ubiquitous promoter up to 500 nt long. Optionally, the ubiquitous promoter comprises the nucleotide sequence of SEQ ID NO: 7. In some aspects, the riAAV comprises an ITR comprising the sequence of SEQ ID NO: 6, SEQ ID NO: 9, or both. In certain aspects, the AAV is an AAV9.

With regard to the presently disclosed methods, in various instances, the subject has CS type B (CSB), or a predisposition thereto. In some aspects, the riAAV comprises a nucleotide sequence encoding a CSB protein, optionally, wherein the nucleotide sequence is a human codon optimized sequence of SEQ ID NO: 2 or comprises the sequence of SEQ ID NO: 14. Optionally, the riAAV is a single-stranded AAV plasmid comprising a truncated poly A sequence and a ubiquitous promoter up to 200 nt long. In various aspects, the truncated poly A sequence comprises a sequence of AATAAA (SEQ ID NO: 4) and a sequence of ACAACATTGCTTCCTAAACTTTCAAGTCCC (SEQ ID NO: 5). In exemplary instances, the riAAV comprises an ITR comprising the sequence of SEQ ID NO: 10, SEQ ID NO: 13, or both. In certain aspects, the AAV is an AAV9.

With regard to the presently disclosed methods, in exemplary aspects, the subject has XPG, or a predisposition thereto. In some aspects, the riAAV comprises a nucleotide sequence encoding an XPG protein, optionally, wherein the nucleotide sequence is a human codon optimized sequence of SEQ ID NO: 3 or comprises the sequence of SEQ ID NO: 12. Optionally, the riAAV comprises a promoter, optionally, wherein the promoter comprises the sequence of SEQ ID NO: 11. In certain aspects, the riAAV comprises an ITR comprising the sequence of SEQ ID NO: 10, SEQ ID NO: 13, or both. In certain aspects, the AAV is an AAV9.

Cells

Further provided herein is a cell comprising any one of the presently disclosed AAV (e.g., riAAV). Optionally, the cell is transduced with the AAV. The cell can be a eukaryotic cell, e.g., plant, animal, fungi, or algae, or can be a prokaryotic cell, e.g., bacteria or protozoa. The cell can be a cultured cell or a primary cell, i.e., isolated directly from an organism, e.g., a human. The cell can be an adherent cell or a suspended cell, i.e., a cell that grows in suspension. In exemplary instances, the cell is preferably a mammalian cell, e.g., a human cell. The cell can be of any cell type, can originate from any type of tissue, and can be of any developmental stage.

Also provided by the invention is a population of cells comprising at least one cell comprising the presently disclosed AAV. The population of cells can be a heterogeneous population. Alternatively, the population of cells can be a substantially homogeneous population, in which the population comprises mainly of cells comprising the AAV. The population also can be a clonal population of cells, in which all cells of the population are clones of a single cell comprising the AAV.

In exemplary aspects, the cell may be transduced with an AAV of the present disclosure and subsequently administered to the subject. The cell may be autologous to the subject receiving the cell or the cell may be allogeneic to the subject. Optionally, the cell is a universal cell which evades recognition by the subject's immune system as “foreign”. Universal cells are known in the art.

Routes of Administration and Doses

In various aspects, the AAV or the cell is provided in a composition (e.g., a pharmaceutical composition) comprising a physiologically-acceptable (i.e., pharmacologically-acceptable) carrier, buffer, excipient, or diluent. Any suitable physiologically-acceptable (e.g., pharmaceutically acceptable) carrier can be used within the context of the disclosure, and such carriers are well known in the art. The choice of carrier will be determined, in part, by the particular site to which the composition is to be administered and the particular method used to administer the composition. The composition also can comprise agents which, for instance, facilitate uptake of the AAV into host cells. Suitable composition formulations include aqueous and non-aqueous solutions, isotonic sterile solutions, which can contain anti-oxidants, buffers, bacteriostats, and solutes that render the formulation isotonic with the blood, and aqueous and non-aqueous sterile suspensions that can include suspending agents, solubilizers, thickening agents, stabilizers, and preservatives.

The composition may be formulated for topical administration (e.g., in the form of aerosol, cream, foam, gel, liquid, ointment, paste, powder, shampoo, spray, patch, disk, or dressing). A “patch” typically includes at least the compositions provided herein and a covering layer, such that, the patch can be placed over an area of skin to be treated. The patch can be designed to maximize delivery of the compositions provided herein through the stratum corneum and into the epidermis or dermis, reduce lag time, promote uniform absorption, and reduce mechanical rub-off.

The composition can be presented in unit-dose or multi-dose sealed containers, such as ampules and vials, and can be stored in a freeze-dried (lyophilized) condition requiring only the addition of the sterile liquid carrier, for example, water, immediately prior to use. A composition comprising AAV or cells comprising AAV is, in one aspect, placed within containers, along with packaging material that provides instructions regarding the use of the composition (i.e., in a kit). Generally, such instructions include a tangible expression describing the reagent concentration, as well as, in certain embodiments, relative amounts of excipient ingredients or diluents (e.g., water, saline or PBS) that may be necessary to reconstitute the composition.

The AAV or cell is administered in an amount and at a location sufficient to provide some improvement or benefit to the subject, e.g., delay the onset of symptoms of CS, increase survival, decrease neurodegeneration, etc. Depending on the circumstances, a composition comprising the AAV or cell is applied or instilled into body cavities, applied directly to target tissue, and/or introduced into circulation. For example, in various circumstances, it will be desirable to deliver the composition by intravenous, intraperitoneal, intracerebral (intra-parenchymal), intramuscular, intra-ocular, intraarterial, intraportal, intralesional, intramedullary, intrathecal, intraventricular, intradermal, intraarticular, intraneuronal, intraganglion, periganglion, transdermal, subcutaneous, intranasal, inhalation (e.g., upper and/or lower airways), enteral, epidural, urethral, vaginal, or rectal means. If desired, the AAV or cell is administered regionally via intramuscular, transdermal, or subcutaneous administration, or intraarterial or intravenous administration feeding the region of interest. In some aspects, the riAAV is administered to the subject intravenously, intraventricularly or intrathecally, or through the cisterna magna, or a combination thereof, optionally, wherein the AAV is administered to the subject intravenously, and at least one of intraventricularly or intrathecally, or through the cisterna magna.

Alternatively, the composition is administered locally via implantation of a membrane, sponge, capsule, or another appropriate material onto which the composition has been absorbed or encapsulated. Where an implantation device is used, the device is, in one aspect, implanted into a suitable tissue, and delivery of the AAV or E-selectin produced by the engineered cell is, for example, via diffusion, timed-release bolus, or continuous administration.

A particular administration regimen for a particular subject will depend, in part, upon the amount of therapeutic administered, the route of administration, and the cause and extent of any side effects. The amount administered to a subject (e.g., a mammal, such as a human) in accordance with the disclosure should be sufficient to affect the desired response over a reasonable time frame. In exemplary aspects, the method comprises administering an initial dose to the subject, optionally, wherein the initial dose is intravenously administered. In optional aspects, the method further comprises administering to the subject one or more subsequent doses of the riAAV at least about 1, 2, 3, 4, or 5 years after the initial dose, at least about 10, 11, 12, 13, 14, or 15 years after the initial dose, at least about 20, 21, 22, 23, 24, or 25 years after the initial dose, or a combination thereof. In exemplary aspects, each dose of riAAV comprises at least or about 5×10¹² vg/kg, at least or about 1×10¹³ vg/kg, at least or about 3×10¹³ vg/kg, or at least or about 3×10¹⁴ vg/kg. Alternatively, other doses may be administered depending on a variety of factors, including, e.g., the severity of disease or symptoms thereof, age, weight, height, of the subject.

Exemplary doses of viral particles in genomic equivalent titers of 10⁴-10¹⁵ transducing units (e.g., 10⁷-10¹² transducing units), or at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, or at least about 10¹⁵ transducing units (e.g., at least about 10⁷, at least about 10⁸, at least about 10⁹, at least about 10¹⁰, at least about 10¹¹, at least about 10¹², at least about 10¹³ or at least about 10¹⁴ transducing units, such as about 10¹⁰ or 10¹² transducing units). In exemplary aspects, the dose of viral particles (VP) per in vitro transduced cell is within about 10³ to about 10¹². In some aspects, the dose of viral particles per in vitro transduced cell is within about 10⁴ to about 10⁸ or about 10⁴ to about 10⁶. For example, the dose of viral particles per in vitro transduced cell is 10⁵ VP/cell. Some conditions require prolonged treatment, which may or may not entail multiple administrations over time.

In exemplary aspects, the dose of the AAV administered to the subject (e.g., via intramuscular injection) is about 50 to about 5000 μl AAV, wherein the concentration of the AAV is within about 10⁸ or 10¹⁶VP/ml. In exemplary aspects, the dose of the AAV administered to the subject (e.g., via intramuscular injection) is about 50 to about 500 μl AAV, wherein the concentration of the AAV is within about 10¹⁰ or 10¹⁴VP/ml. In exemplary aspects, the dose of the AAV administered to the subject (e.g., via intramuscular injection) is about 75 to about 200 μl AAV, wherein the concentration of the AAV is about 10¹² VP/ml.

When appropriate, the AAV or cell is administered in combination with other substances (e.g., therapeutics) and/or other therapeutic modalities to achieve an additional (or augmented) biological effect. This aspect includes concurrent administration (i.e., substantially simultaneous administration) and non-concurrent administration (i.e., administration at different times, in any order, whether overlapping or not) of the AAV or cell and one or more additionally suitable agents(s). It will be appreciated that different components are, in certain aspects, administered in the same or in separate compositions, and by the same or different routes of administration.

According to a further aspect of the disclosure, the AAV or cell is optionally administered separately, sequentially or simultaneously in combination with one or more agents useful for treating the symptoms or causes of CS.

Subjects

In some embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

The following examples are given merely to illustrate the present invention and not in any way to limit its scope.

### EXAMPLES

### Example 1

This example describes Adeno-Associated Virus (AAV)-Mediated Gene Delivery to Treat Xpg−/− Mice.

Cockayne syndrome (CS) is a rare disease characterized by neurodegeneration and premature aging. CS is caused by mutations in various genes involved in DNA repair mechanisms. One subtype of CS is caused by mutations in the Xeroderma Pigmentosum group G (XPG) protein, an endonuclease encoded by the ERCC5 gene. The hybrid C57BL6/FVB F1 Xpg−/− mouse model developed by Drs. Hoeijmakers and Vermeij replicates the CS phenotype well. The overall aims of the project are to: 1) characterize the ability of adeno-associated virus (AAV) mediated gene therapy to prevent development of the CS phenotype in these mice through neonatal administration, and 2) characterize to what extent AAV gene therapy can halt or reverse progression of the disease process following adult administration. To do this, the impact of gene therapy is examined on the hybrid Xpg−/− knockout mouse model following injections of AAV9-ERCC5 using several doses at one of two alternate time-points (neonates or adults). Untreated Xpg−/−, AAV treated Xpg−/−, and WT mice are evaluated weekly by neurological, physical, and behavioral examinations for up to 24 weeks. Mice are euthanized upon reaching a moribund state or at 24 weeks and tissues are harvested for further analyses. Thus far, ERCC5 gene expression has been observed in all AAV-treated groups and the highest dose neonatal treatment cohort has displayed improvements in several functional domains including survival. Further analyses are in progress to evaluate the full potential of gene therapy for CS.

### Example 2

This example describes an exemplary method of treating Cockayne Syndrome.

Cockayne syndrome (CS) is a rare, autosomal recessive disease characterized by neurodegeneration and premature aging. CS is caused by mutations in various genes involved in DNA repair mechanisms. There is no disease-modifying therapy currently available for these patients. One subtype of CS is caused by mutations in the ERCC5 gene that encodes the Xeroderma Pigmentosum group G (XPG) endonuclease protein. The hybrid C57BL6/FVB F1 Xpg−/− mouse model developed by Drs. Hoeijmakers and Vermeij replicates the CS phenotype well. Evaluations in Xpg−/− mice as compared to age and sex matched healthy controls (n=10 per cohort) have revealed lower birthweights, decreased growth, reduced response to incline in negative geotaxis tests at 1 week of age, hindlimb clasping by week 3 (a sign of neurodegeneration), kyphosis development at 9 weeks, tremor development at 10 weeks, and ataxia at 12 weeks. Whole body ActiTrack analyses revealed significant decreases in distance travelled, number of rearings to cross a horizontal plane, and increased time at rest by 3 weeks of age which all became even more dramatic by 12 weeks of age. Xpg−/− mice display growth impairment by 10 weeks of age and reach a moribund state between 14-17 weeks of age.

An adeno-associated virus (AAV)-mediated ERCC5 gene replacement strategy was developed to determine whether this approach can prevent development or reduce severity of the CS phenotype in these mice. AAV9-ERCC5 was intravenously administered to Xpg−/− mouse 1 day old neonates (n 8 per cohort) at one of the following doses (5×10¹², 1×10¹³, 3×10¹³, or 3×10¹⁴ vg/kg) via the temporal vein.

Although most outcome measures were not significantly improved in the two lowest dose treatment groups, tissue biodistribution and gene expression analyses did reveal approximately 1.5- to 2-fold increased expression in all tissues assessed. Both low dose treatment groups also showed on average 3 to 4 weeks of increased survival as compared to untreated controls and brain histological analyses demonstrate normalization of nucleus sizes in treated mice which suggests some improvement in the underlying DNA repair disorder.

Studies to fully evaluate the two higher treatment cohorts are ongoing. Currently (at 15 weeks of age), no mice from the higher dose treatment cohorts have displayed any signs of tremors, kyphosis, or ataxia, and none have reached a moribund state. ActiTrack analyses have revealed normalization of distance travelled, rearings, and resting times in both of the high dose treatment cohorts at 12 weeks of age suggesting significant improvements in whole body activity ability. Evaluations are to continue to 24 weeks of age at which time full necropsies are performed for further analyses of vector genome biodistribution, ERCC5 transcription, histological evaluations, and other protein studies. In summary, the data support gene replacement strategies to treat Cockayne Syndrome.

### Example 3

This example describes the development of AAV-based therapy for the treatment of CS in humans.

The preceding examples relate to one subtype of CS which is caused by mutations in the Xeroderma Pigmentosum group G (XPG) protein, an endonuclease encoded by the ERCC5 gene. Although this subtype is among the rarest versions of CS in humans, the hybrid C57BL6/FVB F1 Xpg^{-/-} mouse model developed by Drs. Hoeijmakers and Vermeij replicates the CS phenotype more accurately than other CS mouse models. The overall aim of this study was to determine whether adeno-associated virus (AAV) mediated gene therapy could prevent or lessen the development of the CS phenotype in these mice through neonatal administration.

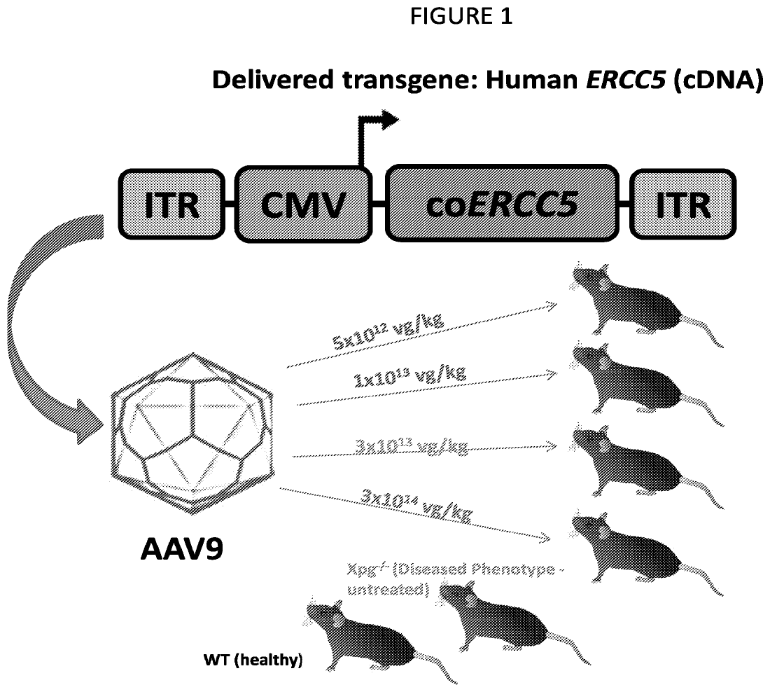

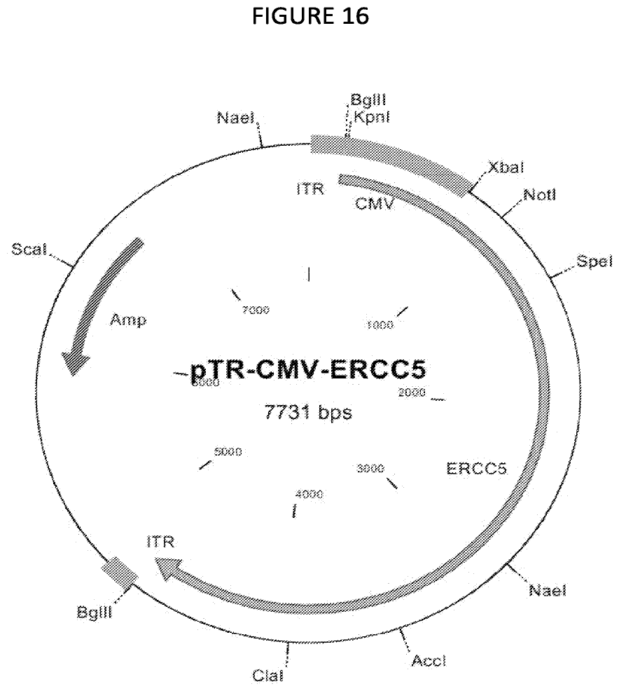

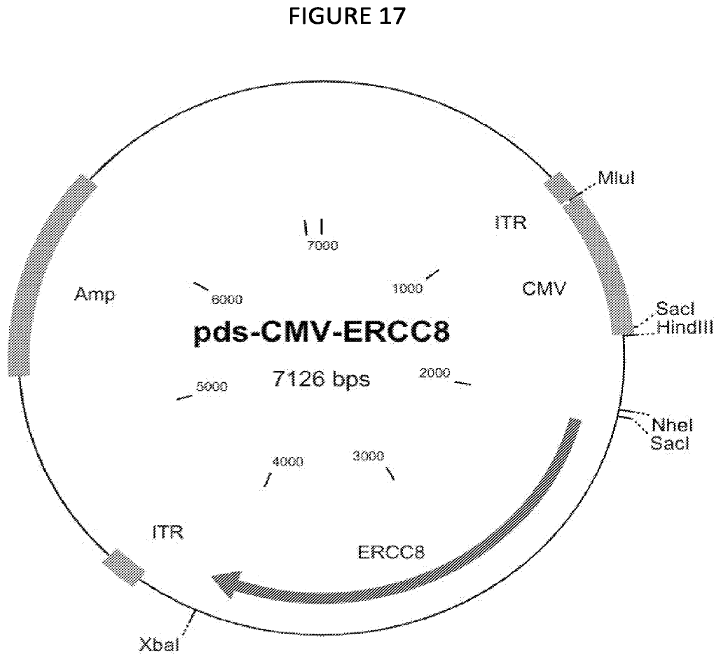

In Examples 1 and 2, the impact of gene therapy on the hybrid Xpg^{-/-} knockout mouse model was evaluated following intravenous injections of AAV9-ERCC5 using several doses and compared to WT healthy mice. A vector map of the AAV9-ERCC5 used in this study is shown in FIG. 16. A codon optimized ERCC5 sequence (SEQ ID NO: 12) is cloned into an AAV9 comprising ITRs of SEQ ID NOs: 10 and 13 and a CMV promoter of SEQ ID NO: 11. WT mice or the hybrid Xpg^{-/-} knockout mice were either untreated or treated with the AAV9-ERCC5. If treated, one of the following doses was administered: 5×10¹² vg/kg, 1×10¹³ vg/kg, 3×10¹³ vg/kg, or 3×10¹⁴ vg/kg. See FIG. 1.

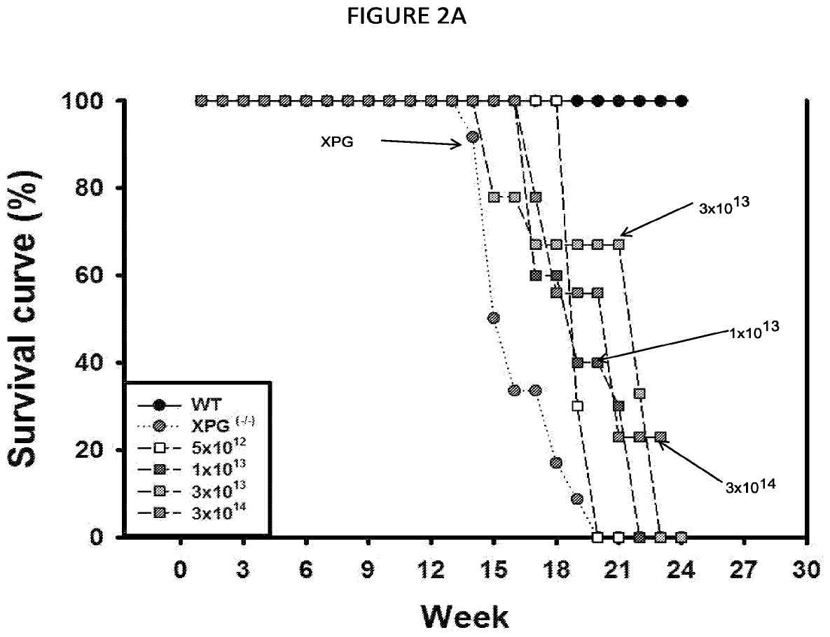

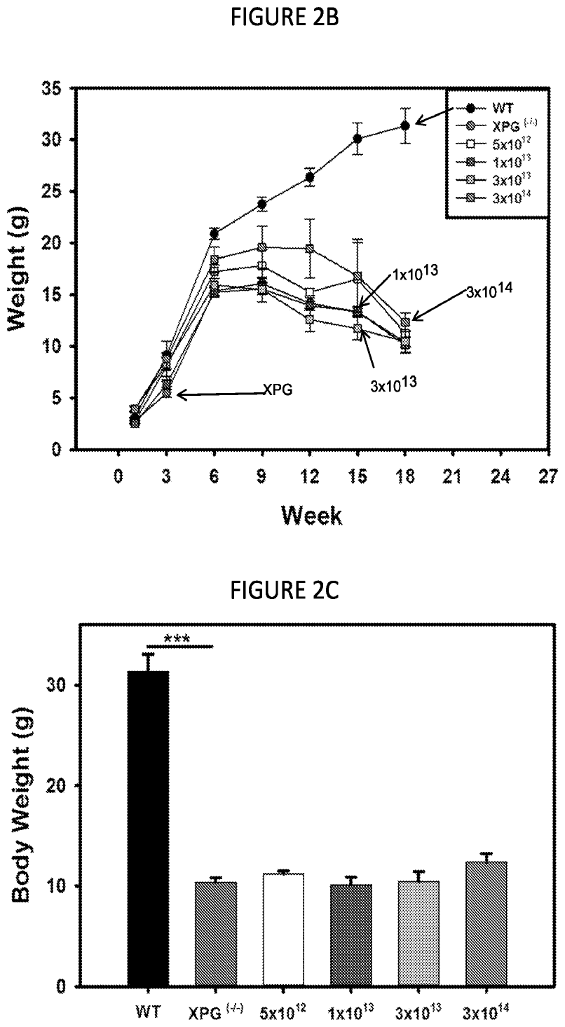

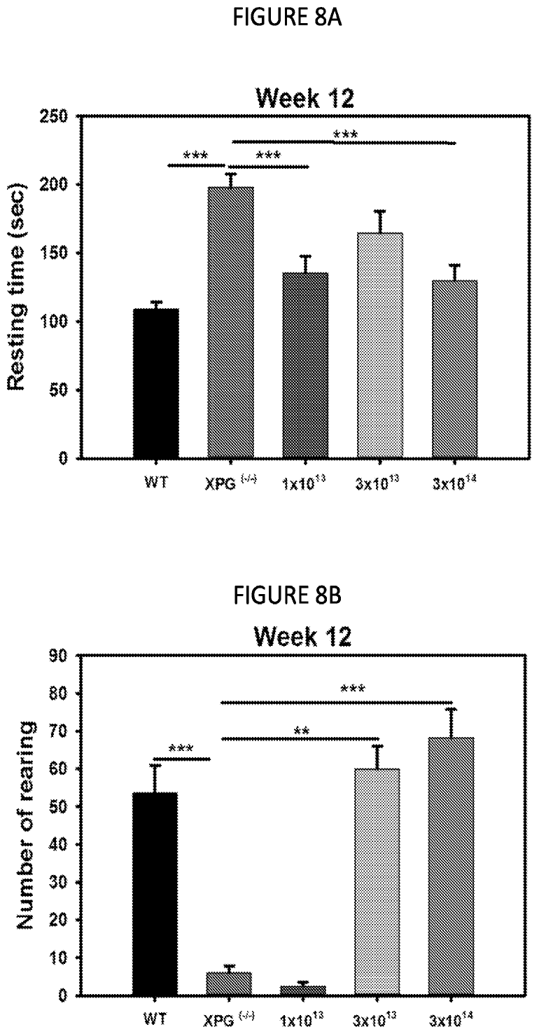

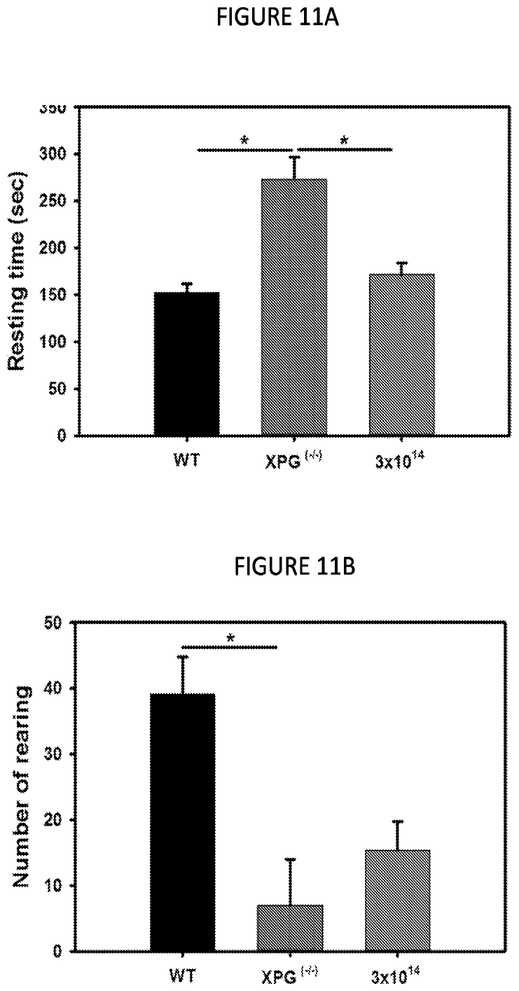

Untreated Xpg^{-/-}, AAV treated Xpg^{-/-}, and WT mice were evaluated weekly by neurological, physical, and behavioral examinations for up to 24 weeks. The results are shown in FIGS. 2-11. Briefly, the mice were measured for viability prior to reaching a moribund state (at which point they were euthanized) as a measure of survival. As shown in FIG. 2A, the % survival for Xpg^{-/-} mice treated with AAV9-ERCC5 was generally higher relative to the untreated Xpg^{-/-} mice. Twenty percent of mice given the highest dose of AAV9-ERCC5 survived to 23 weeks, whereas 0% of untreated Xpg^{-/-} mice survived to 20 weeks. Interestingly, almost 40% of mice given the second-highest dose of AAV9-ERCC5 survived past 21 weeks. About 35% of mice given the third-highest dose of AAV9-ERCC5 survived past 21 weeks. On average, each higher dose extended survival by an additional 1-2 weeks.

Body weights of mice were measured throughout the study. As shown in FIG. 2B, the weights of all Xpg^{-/-} mice were less than the body weights of WT healthy mice. However, mice given the highest dose of AAV9-ERCC5 had a less significant weight loss than that observed in untreated mice. FIG. 2C demonstrates the body weights of mice at Week 18 of the study.

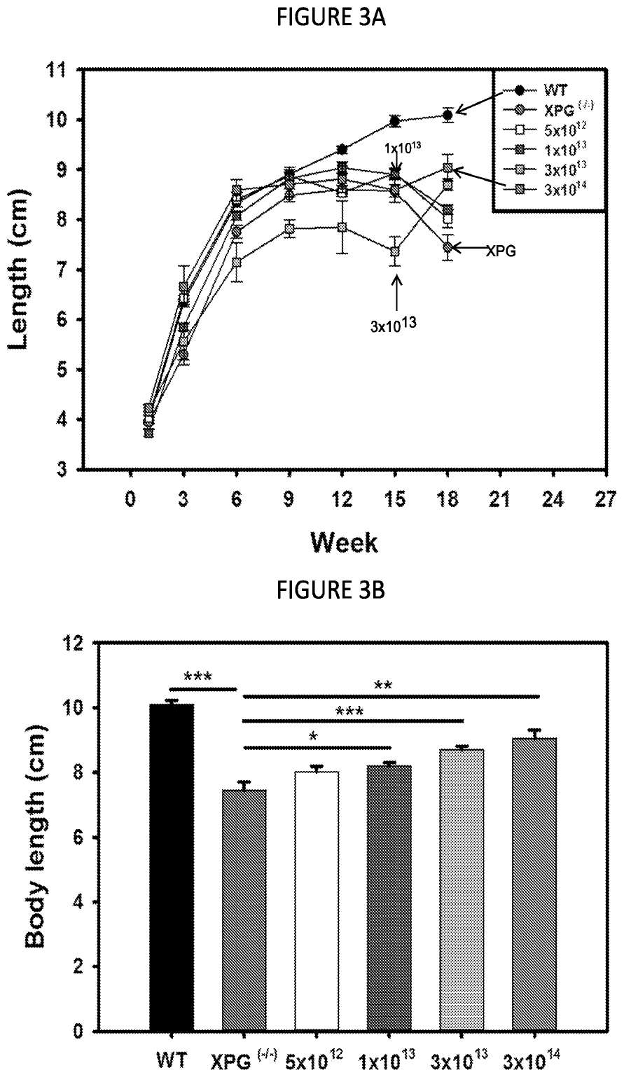

Body lengths also were measured throughout the study. As shown in FIG. 3A, the lengths of all Xpg^{-/-} mice were less than the body lengths of WT healthy mice. However, at Week 18, mice treated with any dose of AAV9-ERCC5 were longer in length relative to untreated mice (FIG. 3B).

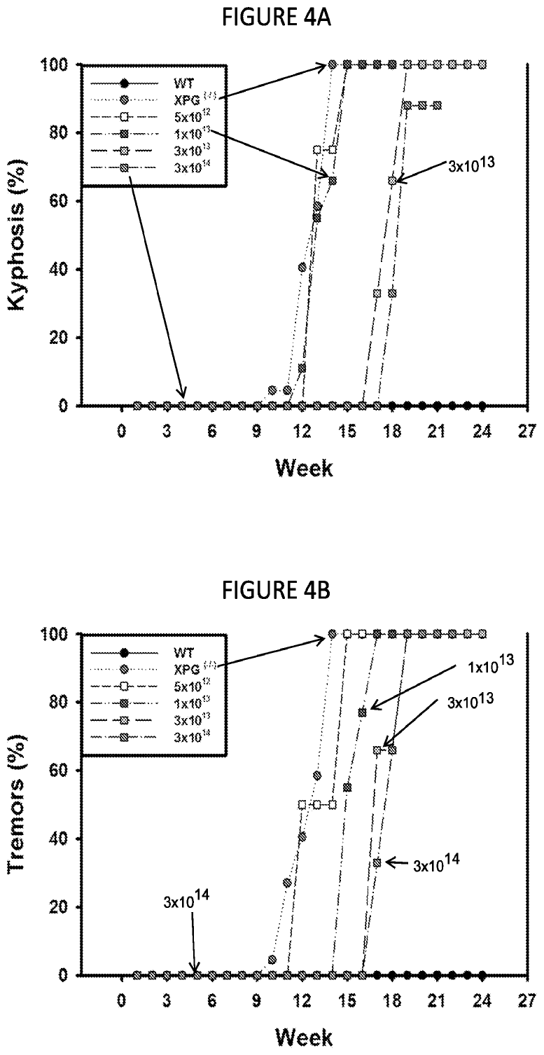

Development of the CS symptoms kyphosis (an exaggerated, forward rounding of the back) and tremors were determined by blinded observation throughout the study as a binary assessment of either present or not. As shown in FIGS. 4A and 4B, the 100% of untreated Xpg^{-/-} mice exhibited these CS symptoms by Week 15, whereas, at this timepoint 0% of mice treated with the highest dose of AAV9-ERCC5 demonstrated kyphosis and tremors. Treatment delayed the onset of kyphosis and tremors. FIGS. 4A and 4B.

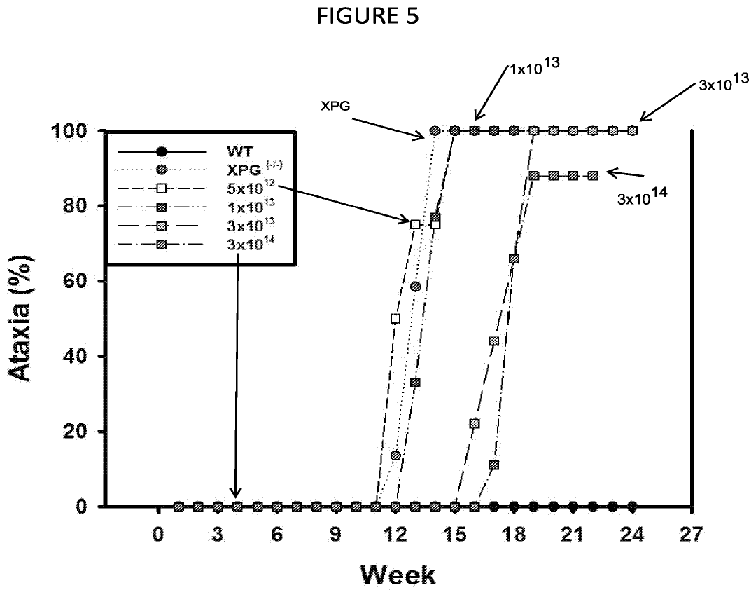

Ataxia and hind-limb clasping were also measured throughout the study. As shown in FIG. 5, 100% of untreated mice exhibited ataxia around Week 13, whereas at this timepoint, 0% of mice treated with the highest dose or second highest dose of AAV9-ERCC5 demonstrated ataxia, and less than 40% of mice treated with the third highest dose demonstrated ataxia. Even mice given the lowest dose demonstrated less than 80% ataxia at Week 13.

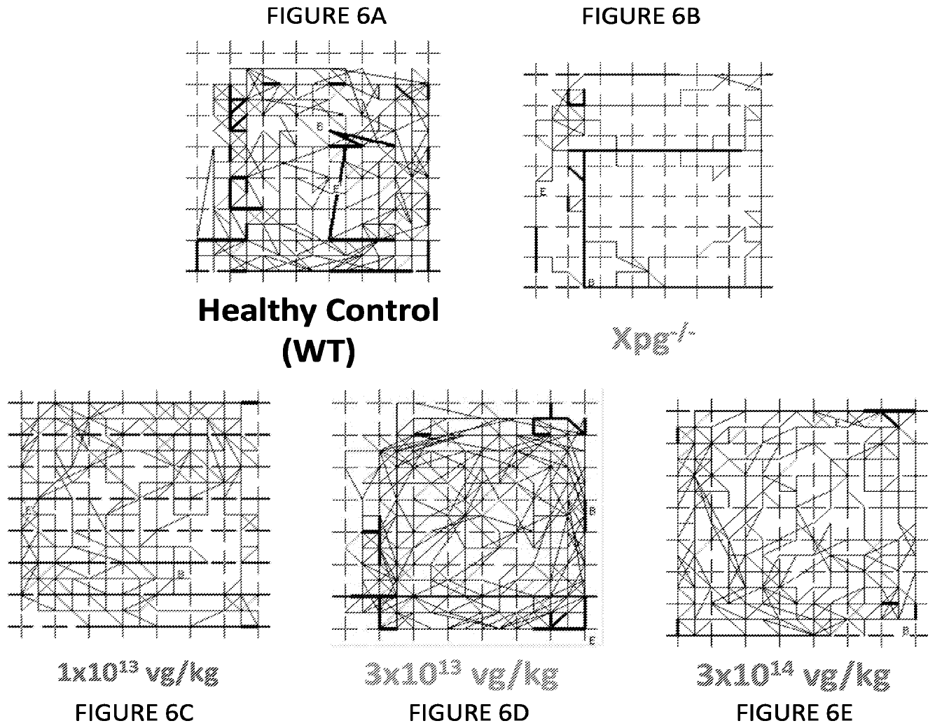

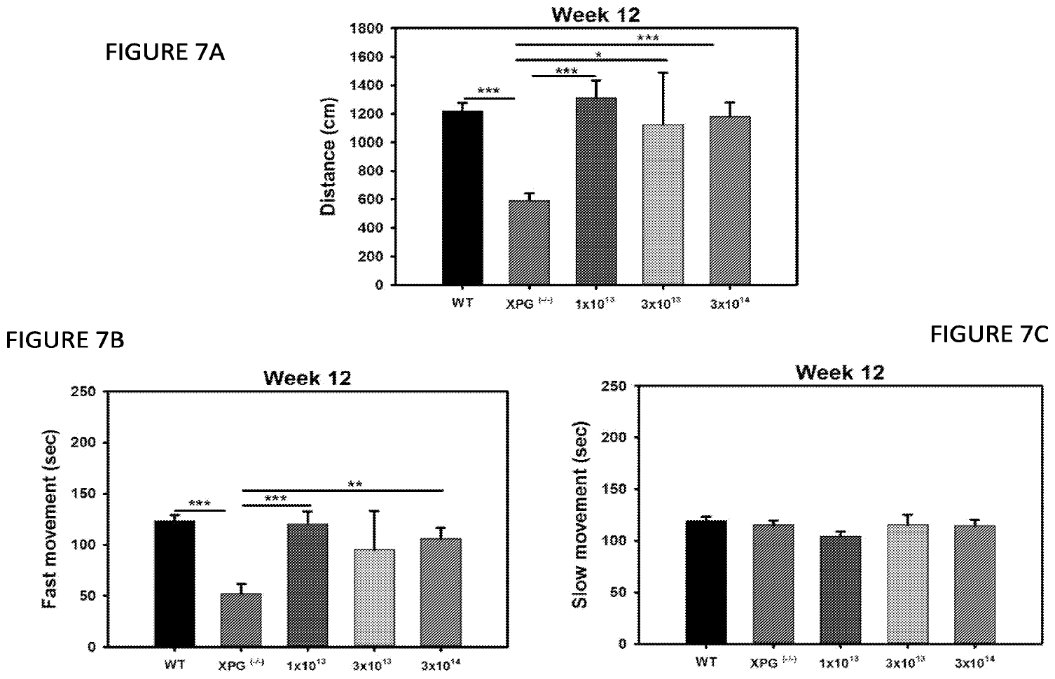

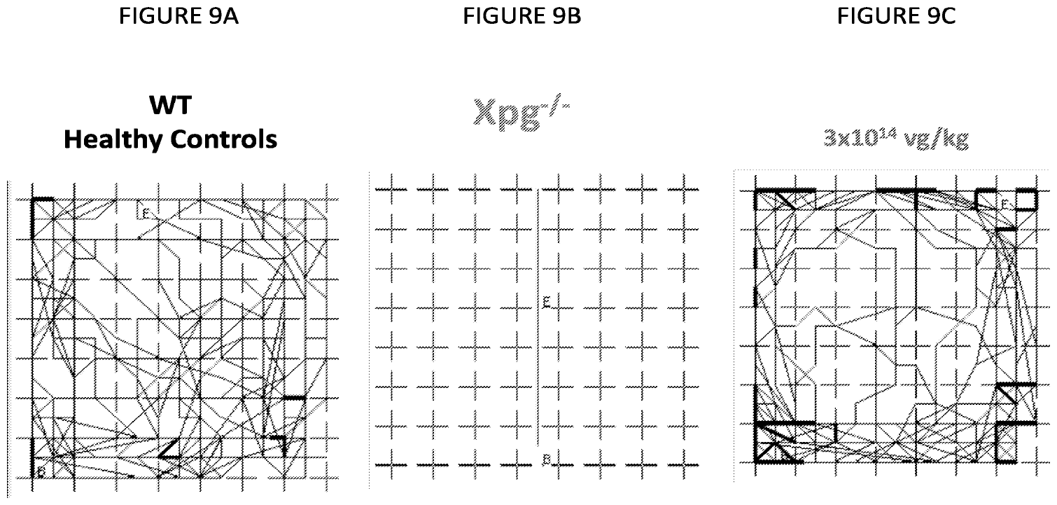

Whole body functional evaluations were carried out using an ActiTrack system (Panlab software). Briefly, mice were placed in the detection chamber to assess total distance and vertical activity for 6 minutes prior to exercise. These assessments are the pre-exercise assessments presented in FIGS. 6-8. Next, the mice were mildly exercised on a treadmill (5 min at 3 m/min followed by an increase to 10 m/min for 10 min). Post-treadmill activity was then measured using the same method described for pre-treadmill activity and is presented in FIGS. 9-11. Zone map tracings were created using the Actitrack mapping software where the x and y planes represent the two dimensions of the activity chamber, and the bold lines represent every time the mouse breaks the z plane, registering as vertical activity within each 6 min time interval (pre- and postexercise). FIGS. 6A-6E demonstrate Week 12 activity in the treated and untreated cohorts. WT healthy controls are also shown. As shown in these figures, mice that were treated with AAV9-ERCC5 were more active compared to the untreated mice. Distance and fast and slow movements were measured at Week 12 and the results are shown in FIGS. 7A-7C. As shown in FIG. 7A, the treated mice moved greater distances and demonstrated longer amounts of fast movements. A fast movement was considered as a movement of greater than or equal to 5 cm/second. Slow movements were about the same among all groups. Based on these data, it was suggested that the treatment allowed for increased distance and speed.

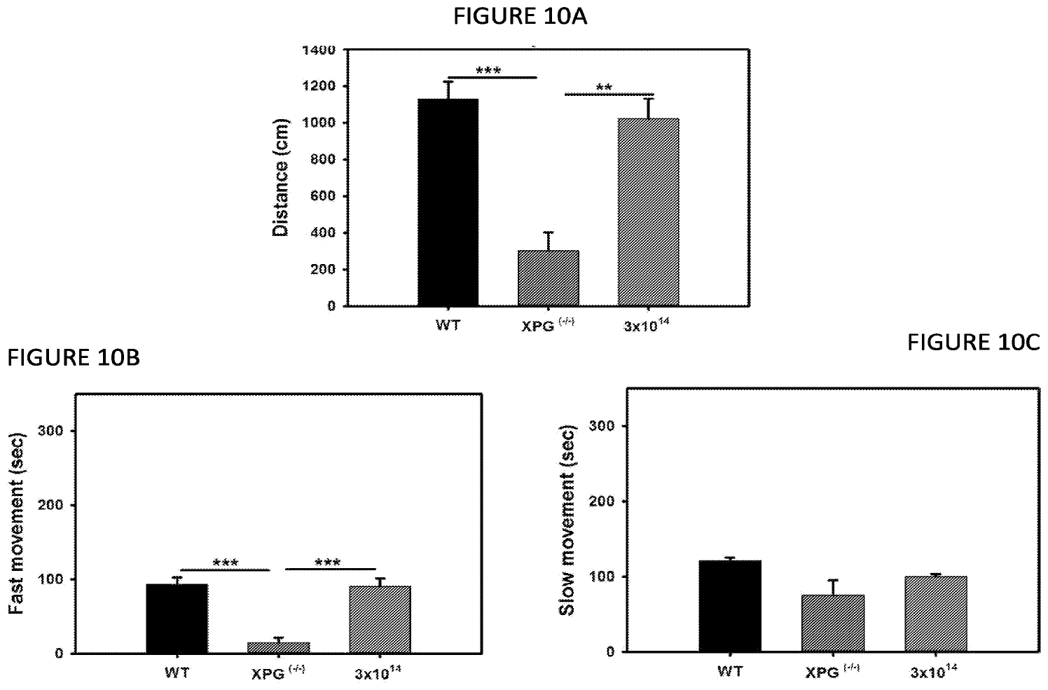

Resting and rearing were measured using the ActiTrack system, too. After exercise on a treadmill, movements, distance, and speed were measured. Each of these (movements, distance and speed) were greatest for mice given the highest dose of AAV9-ERCC5. FIGS. 9A-9C and 10A-10C. Post-exercise resting and rearing were tested and for mice treated with the highest dose, weakness was revealed in this group as the mice were not as likely to rear following exercise as the healthy controls. These data support the pursuit of re-administration and/or the administration of gene replacement therapy to multiple routes.

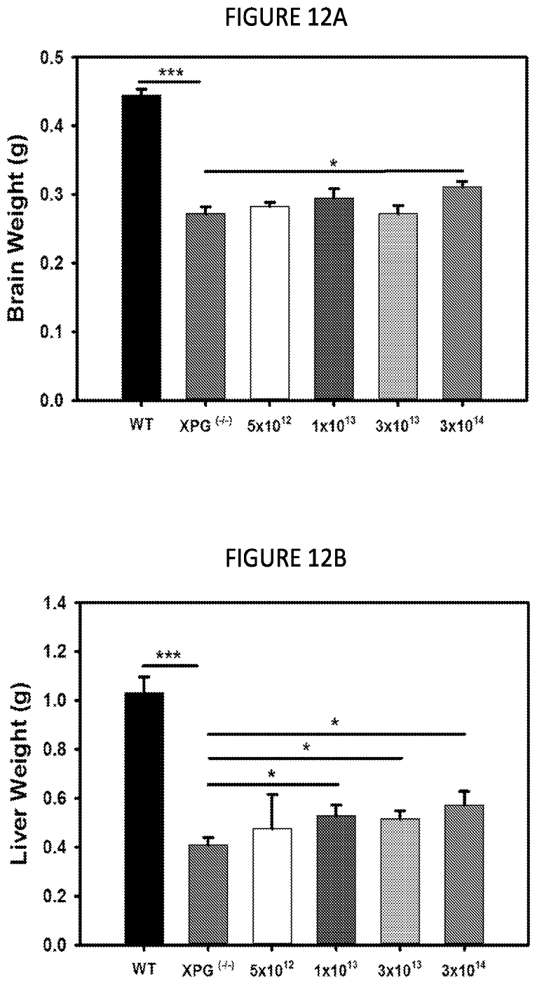

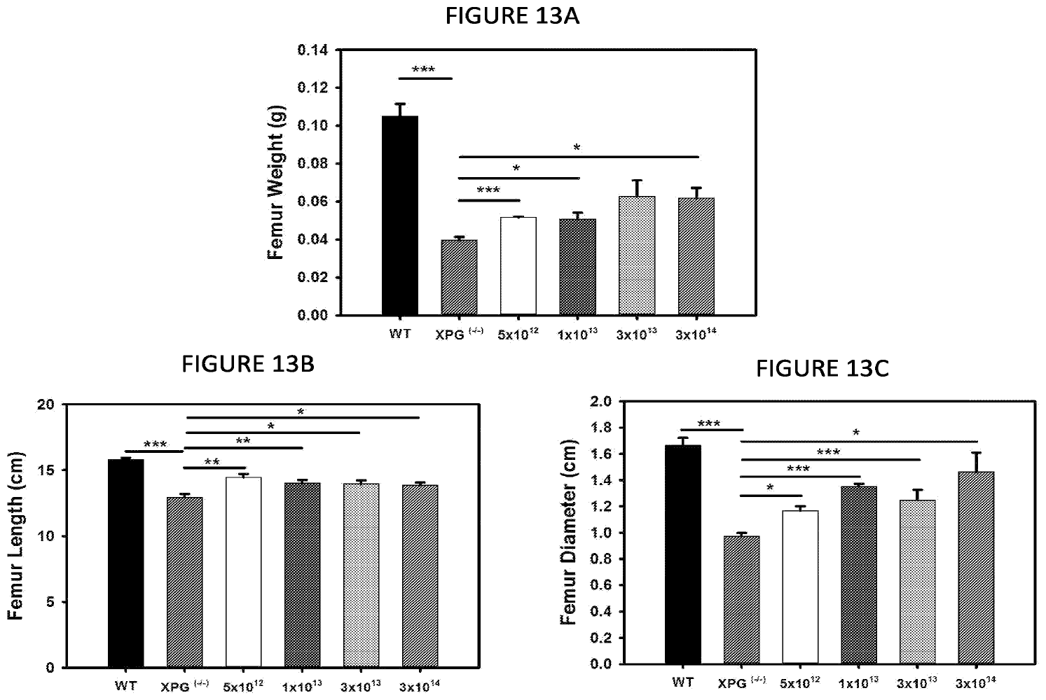

Mice were euthanized upon reaching a moribund state or at 24 weeks and tissues are harvested for further analyses. Femur weight, length and diameter were measured (FIGS. 13A-13C) as well as brain and liver weights (FIGS. 12A-12B). The measurements for WT healthy mice were taken at 18-24 weeks whereas measurements were taken at Weeks 12-21 for Xpg^{-/-} mice. As shown in FIGS. 12A and 12B, brain and liver weights were about the same for each of the Xpg^{-/-} mice. As shown in FIG. 13A, femur weights and femur diameters increase as dose amounts increased, though each weight and diameter of Xpg^{-/-} mice was less than that of WT healthy mice. Femur lengths were about the same among all groups of mice. Results are shown in FIGS. 12-15.

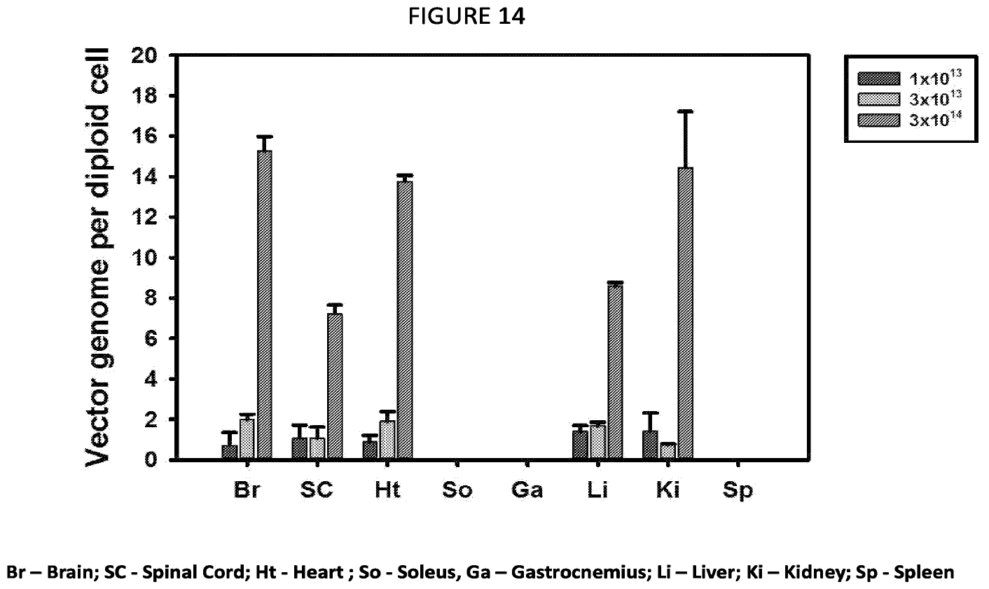

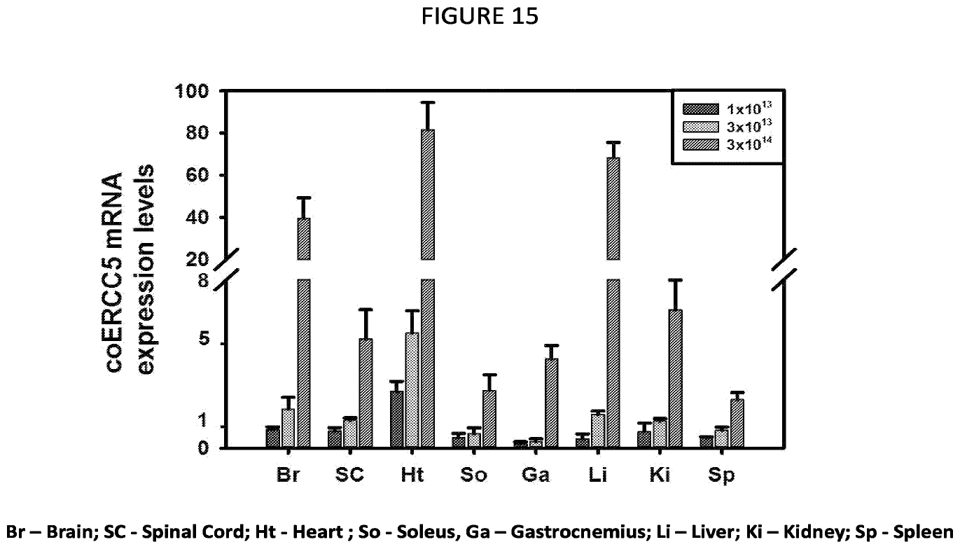

Vector biodistribution was measured in several tissues, including brain, spinal cord, heart, soleus, liver, kidney, spleen and gastrocnemius. As shown in FIGS. 14-15, the highest expression of ERCC5 mRNA were found in the brain, heart, and liver.

This study demonstrated that AAV9-mediated gene delivery of the ERCC5 gene led to gene expression. The highest dose treatment cohort displayed improvements in several functional domains including survival. The treated mice demonstrated improved quality of life and a delayed onset of neurodegeneration.

### Example 4

This example describes the generation of an AAV9 comprising a human codon optimized version of ERCC8 to test for treatment of subjects with CSA.

Mice with a knock-out (KO) of the ERCC8 gene are made and the phenotype of such mice are characterized relative to WT healthy mice. The characterization includes the measurements described in Example 3. The KO mice and then used to test therapeutic efficacy of an AAV9 comprising a codon optimized ERCC8 sequence. A vector map of the AAV9 is provided in FIG. 16. The ERCC8 gene is 1188 nucleotides long and is cloned into a double-stranded AAV plasmid containing the standard SV40 poly A sequence for testing with ubiquitous promoters that can be up to 500 nucleotides in length. The first ITR comprises the sequence of SEQ ID NO: 6 and the second ITR comprises the sequence of SEQ ID NO: 9. The codon optimized ERCC8 comprises the sequence of SEQ ID NO: 8 and the CMV promoter comprises the sequence of SEQ ID NO: 7.

The KO mice are treated with similar doses described above in Example 3 or left untreated. One cohort of healthy WT mice are used in the study as a control Untreated Xp^{-/-}, AAV treated Xpg^{-/-}, and WT mice were evaluated weekly by neurological, physical, and behavioral examinations for up to 24 weeks, as essentially described in Example 3.

Delayed onset of symptoms of CSA is expected in mice treated with the AAV9-ERCC8, relative to untreated KO mice.

### Example 5

This example describes the generation of an AAV9 comprising a human codon optimized version of ERCC6 to test for treatment of subjects with CSB.

Mice with a knock-out (KO) of the ERCC6 gene are made and the phenotype of such mice are characterized relative to WT healthy mice. The characterization includes the measurements described in Example 3. The KO mice and then used to test therapeutic efficacy of an AAV9 comprising a codon optimized ERCC6 sequence. The sequence of the codon optimized ERCC6 is provided as SEQ ID NO: 14. The ITRs are provided as SEQ ID NOs: 10 and 13. Due to its length the codon optimized ERCC6 is cloned into a single-stranded AAV plasmid containing a truncated poly A comprising the canonical AATAAA (SEQ ID NO: 4) plus ^{˜}30 bp of natural ERCC6 3′ UTR sequence past the stop codon (SEQ ID NO: 5). The promoter used in the vector is one which is 200 nucleotides or less in length.

The KO mice are treated with similar doses described above in Example 3 or left untreated. One cohort of healthy WT mice are used in the study as a control Untreated Xpg^{-/-}, AAV treated Xpg^{-/-}, and WT mice were evaluated weekly by neurological, physical, and behavioral examinations for up to 24 weeks, as essentially described in Example 3.

Delayed onset of symptoms of CSB is expected in mice treated with the AAV9-ERCC6, relative to untreated KO mice.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. Further, the disclosure contemplates use of variants of the sequences provided in the Sequence Listing, including sequences having at least 80%, at least 85%, at least 90% or having greater than 90% sequence identity (e.g., about 91%, about 92%, about 93%, about 94%, about 95%, about 96%, about 97%, about 98%, about 99%) to an amino acid sequence or nucleic acid set forth in the Sequence Listing.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A method of treating a subject with Cockayne Syndrome (CS) or a predisposition thereto, comprising administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to treat CS in the subject.

2. A method of treating a subject with one or more mutations in an ERCC5 gene, an ERCC8 gene, an ERCC6 gene, or a combination thereof, comprising administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to treat CS in the subject.

3. A method of delaying the onset of Cockayne Syndrome (CS), or a symptom thereof, in a subject with one or more mutations in an ERCC5 gene, an ERCC8 gene, an ERCC6 gene, or a combination thereof, comprising administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to delay the onset of CS or the symptom thereof in the subject.

4. The method of claim 3, wherein the symptoms of CS is neurodegeneration, tremors, dystonia, ataxia, hearing loss, vision loss, cataracts, cognitive disability, cachexia (failure to thrive), severe growth defects (short stature), microcephaly, kyphosis, skin photosensitivity, liver failure, renal dysfunction, or a combination thereof.

5. A method of slowing, halting or reversing progression of Cockayne Syndrome (CS) in a subject, comprising administering to the subject a replication-incompetent Adeno-associated Virus (riAAV) comprising a nucleotide sequence encoding a Xeroderma Pigmentosum group G (XPG) protein, a Cockayne Syndrome type A (CSA) protein, or a Cockayne Syndrome type B (CSB) protein in an amount effective to slow, halt or reverse the progression in the subject.

6. The method of any one of the preceding claims, wherein the XPG protein is the protein encoded by the human ERCC5 gene.

7. The method of any one of the preceding claims, wherein the CSA protein is the protein encoded by the human ERCC8 gene.

8. The method of any one of the preceding claims, wherein the CSB protein is the protein encoded by the human ERCC6 gene.

9. The method of any one of the preceding claims, wherein the subject has one or more mutations in the ERCC5 gene, the ERCC8 gene, and/or the ERCC6 gene, optionally, wherein the subject is at least heterozygous for at least one causative mutation in at least one of SEQ ID NOs: 1-3.

10. The method of any one of the preceding claims, wherein the subject has CS type A (CSA), or a predisposition thereto.

11. The method of claim 9 or 10, wherein the riAAV comprises a nucleotide sequence encoding a CSA protein, optionally, wherein the nucleotide sequence is a human codon optimized sequence of SEQ ID NO: 1 or comprises the sequence of SEQ ID NO: 8.

12. The method of claim 11, wherein the riAAV is a double-stranded AAV plasmid comprising an SV40 poly A sequence and a ubiquitous promoter up to 500 nt long.

13. The method of claim 11 or 12, wherein the ubiquitous promoter comprises the nucleotide sequence of SEQ ID NO: 7.

14. The method of any one of claims 11-13, wherein the riAAV comprises an ITR comprising the sequence of SEQ ID NO: 6, SEQ ID NO: 9, or both.

15. The method of any one of the preceding claims, wherein the subject has CS type B (CSB), or a predisposition thereto.

16. The method of claim 9 or 15, wherein the riAAV comprises a nucleotide sequence encoding a a CSB protein, optionally, wherein the nucleotide sequence is a human codon optimized sequence of SEQ ID NO: 2 or comprises the sequence of SEQ ID NO: 14.

17. The method of claim 16, wherein the riAAV is a single-stranded AAV plasmid comprising a truncated poly A sequence and a ubiquitous promoter up to 200 nt long.

18. The method of claim 16 or 17, wherein the truncated poly A sequence comprises a sequence of AATAAA (SEQ ID NO: 4) and a sequence of ACAACATTGCTTCCTAAACTTTCAAGTCCC (SEQ ID NO: 5).

19. The method of any one of claims 16-18, wherein the riAAV comprises an ITR comprising the sequence of SEQ ID NO: 10, SEQ ID NO: 13, or both.

20. The method of any one of the preceding claims, wherein the subject has XPG, or a predisposition thereto.

21. The method of claim 20, wherein the riAAV comprises a nucleotide sequence encoding an XPG protein, optionally, wherein the nucleotide sequence is a human codon optimized sequence of SEQ ID NO: 3 or comprises the sequence of SEQ ID NO: 12.

22. The method of claim 20, wherein the riAAV comprises a promoter, optionally, wherein the promoter comprises the sequence of SEQ ID NO: 11.

23. The method of any one of claims 21-22, wherein the riAAV comprises an ITR comprising the sequence of SEQ ID NO: 10, SEQ ID NO: 13, or both.

24. The method of any one of the preceding claims, wherein the riAAV is an AAV9 serotype.

25. The method of any one of the preceding claims, wherein the riAAV is administered to the subject intravenously, intraventricularly or intrathecally, or through the cisterna magna, or a combination thereof, optionally, wherein the AAV is administered to the subject intravenously, and at least one of intraventricularly or intrathecally, or through the cisterna magna.

26. The method of any one of the preceding claims, comprising administering an initial dose to the subject, optionally, wherein the initial dose is intravenously administered.

27. The method of claim 26, further comprising administering to the subject one or more subsequent doses of the riAAV at least about 1, 2, 3, 4, or 5 years after the initial dose, at least about 10, 11, 12, 13, 14, or 15 years after the initial dose, at least about 20, 21, 22, 23, 24, or 25 years after the initial dose, or a combination thereof.

28. The method of any one of the preceding claims, wherein each dose of riAAV comprises at least or about 5×10¹² vg/kg, at least or about 1×10¹³ vg/kg, at least or about 3×10¹³ vg/kg, or at least or about 3×10¹⁴ vg/kg.

29. A replication-incompetent Adeno-associated Virus serotype 9 (riAAV9) comprising a human codon optimized ERCC5 gene, optionally, SEQ ID NO: 8, and comprising a promoter of SEQ ID NO: 7, a mutated double-stranded ITR of SEQ ID NO: 6 and an ITR of SEQ ID NO: 9.

30. An replication-incompetent Adeno-associated Virus serotype 9 (riAAV9) comprising a human codon optimized ERCC6 gene, optionally, SEQ ID NO: 14 a truncated poly A sequence comprising a sequence of SEQ ID NO: 4 and a sequence of SEQ ID NO: 5, a ubiquitous promoter up to 200 nt long, an ITR of SEQ ID NO:10 and an ITR of SEQ ID NO: 13, wherein the AAV is a single-stranded AAV plasmid.

31. An replication-incompetent Adeno-associated Virus serotype 9 (riAAV9) comprising a human codon optimized ERCC5 gene, optionally, SEQ ID NO: 12, a promoter comprising SEQ ID NO: 11, an ITR of SEQ ID NO:10 and an ITR of SEQ ID NO: 13.

32. A human cell comprising the AAV of any one of claims 29-31.

33. Use of the human cell of claim 32 for treating a subject with Cockayne Syndrome (CS) or a predisposition thereto, or delaying the onset of Cockayne Syndrome (CS), or symptoms thereof, in a subject with one or more mutations in an ERCC gene.

34. Use of the human cell of claim 32 for slowing, halting or reversing progression of Cockayne Syndrome (CS) in a subject.


SEQUENCE LISTING (17 sequences)

SEQ ID NO: 1 - DNA, 2011 bp, Homo Sapiens
  ref <308>: DATABASE ACCESSION NUMBER: GENBANK / U28413.1
  ref <309>: DATABASE ENTRY DATE: 1995-12-02
  ref <313>: RELEVANT RESIDUES IN SEQ ID NO: (1)..(2011)
cgacgtccagtgctccagccggtgtgaggacacgatatgctggggtttttgtccgcacgccaaacgggtttggaggaccctcttcgccttcggagagcagagtcaacacggagagttttgggactggaattaaataaagacagagatgttgaaagaatccacggcggtggaattaacacccttgacattgaacctgttgaagggagatacatgttatcaggtggttcagatggtgtgattgtactttatgaccttgagaactccagcagacaatcttattacacatgtaaagcagtgtgttccattggcagagatcatcctgatgttcacagatacagtgtggagactgtacagtggtatcctcatgacactggcatgttcacatcaagctcatttgataaaactctgaaagtatgggatacaaatacattacaaactgcagatgtatttaattttgaggaaacagtttatagtcatcatatgtctccagtctccaccaagcactgtttggtagcagttggtactagaggacccaaagtacaactttgtgacttgaagtctggatcctgttctcacattctacagggtcacagacaagaaatattagcagtttcctggtctccacgttatgactatatcttggcaacagcaagtgctgacagtagagtaaaattatgggatgtgagaagagcatcaggatgtttgattactcttgatcaacataatgggaaaaagtcacaagctgttgaatcagcaaacactgctcataatgggaaagttaatggcttatgttttacaagtgatggacttcacctcctcactgttggtacagataatcgaatgaggctctggaatagttccaatggagaaaacacacttgtgaactatggaaaagtttgtaataacagtaaaaaaggattgaaattcactgtctcctgtggctgcagttcagaatttgtttttgtaccatatggtagcaccattgctgtttatacagtttactcaggagaacagataactatgcttaagggacattataaaactgttgactgctgtgtatttcagtcaaatttccaggaactttatagtggtagcagagactgcaacattctggcttgggttccatccttatatgaaccagttcctgatgatgatgagactacaacaaaatcacaattaaatccggcctttgaagatgcctggagcagcagtgatgaagaaggatgaatatcatctttagtacctttttgtctctgctgaaactttttaaatgagactgtgtttttttcaactgtatggtctattcctgacagctaaattagccctaaatgcgggtaatatttttcctcatgttttaaaatgaggttaatatttgcataaaatcctaaaacagacttctgtatagtttatttagtcaaaatgtgttccttgatcccagatgttgtggcctgggaaagccctcattgctacagtacaagtaacacaagtcgttgtacctcagttgtgaccttcagcagattttatgaactataagatgcagtctcagaggatcagcaagtggaggccatcagtattgactttctcttacttgctgtactatcagcctgctcgtttccacctttaagaatgattttgccaagaatgattatatcaaaaatagtagttgaaatggtaacatcaaaattattttattctttcttcttcatgtattcacatttttcagtggtttcatttaattaaccatgctttatgttaaacattttggggctcaatgtctcctactatccaaaatgtgcatcacaggaggctcttaactttgtgaaaatcccatgtttgctttattttattttaatgtcagaaggcagtttgcgctaatgcttgaactctttttctgtgaaactcattaaggtatgaccaaatcctgcctcattaattcaagcagaaaatatcctggcagggaatctggcttaaacatgaaatgctgtaataaaatttctatgttattgtctca

SEQ ID NO: 2 - DNA, 4714 bp, Homo Sapiens
  ref <308>: DATABASE ACCESSION NUMBER: Genbank / L04791.1
  ref <309>: DATABASE ENTRY DATE: 1994-11-08
  ref <313>: RELEVANT RESIDUES IN SEQ ID NO: (1)..(4714)
tgggttccaaggcggctggcggcggtagcgtctctgtttccttgtgggcgctcgcgcggccctgggtagtctgtagagaatgccaaatgagggaatcccccactcaagtcaaactcaggagcaagactgtttacagagtcaacctgtcagtaataatgaagaaatggcaatcaagcaagaaagtggtggtgatggggaggtggaggagtacctgtcctttcgttctgtgggtgacgggctgtccacctctgctgtggggtgcgcatcagcagctccgaggagagggccagccctgctgcacatcgaccgacatcagatccaggcagtagagcctagcgcccaggcccttgagctgcagggtttgggtgtggacgtctatgaccaggacgtgctggaacagggagtgcttcagcaggtggacaatgccatccatgaggccagccgtgcctcccagctcgttgacgtggagaaggagtatcggtcggtcctggatgacctcacgtcatgtacgacatccctaaggcaaatcaataaaattattgaacagcttagccctcaagctgccaccagcagagacatcaacaggaaactagattctgtaaaacgacagaagtataataaggaacaacagctaaaaaagatcactgcaaaacaaaagcatctccaggccatccttggaggagcagaggtgaaaattgaactagatcacgccagtctggaggaggatgcagagccggggccatccagtcttggcagcatgctcatgcctgtccaggagactgcctgggaagagctcatccgcactggccagatgacaccttttggtacccagatccctcagaaacaggagaaaaagcccagaaaaatcatgcttaatgaagcatcaggcttcgaaaagtatttggcagatcaagcaaaactgtcttttgaaaggaagaagcaaggttgtaataaaagagcagctagaaaagctccagccccagtcacgcctccagccccagtgcaaaataaaaacaaaccaaacaagaaagccagagttctgtccaaaaaagaggagcgtttgaaaaagcacatcaagaaactccagaagagggctttgcagttccaggggaaagtgggattgccaaaggcaaggagaccttgggagtcagacatgaggccagaggcagagggagactctgagggtgaagagtctgagtatttccccacagaggaggaggaagaggaggaagatgacgaggtggagggggcagaggcggacctgtctggagatggtactgactatgagctgaagcctctgcccaagggcgggaaacggcagaagaaagtgccagtgcaggagattgatgatgactttttcccaagttctggggaagaagctgaagctgcttctgtaggagaaggaggaggaggaggtcggaaagtgggaagataccgagatgatggagatgaagattattataagcagcggttaaggagatggaataaactgagactgcaggacaaagagaaacgtctgaagctggaggacgattctgaggaaagtgatgctgaatttgacgaaggttttaaagtgccaggttttctgttcaaaaagctttttaagtaccagcagacaggtgttaggtggctgtgggaattgcactgccagcaggcaggaggaattctgggagatgaaatgggattgggcaagaccatccagataattgccttcttggcaggtctgagctacagcaagatcaggactcgtggttcaaattacaggtttgaggggttgggtccaactgtaattgtctgtccaacaacagtgatgcatcagtgggtgaaggaatttcacacgtggtggcctccgttcagagtggcaattctacatgaaaccggttcctatacccacaaaaaggagaaactaattcgagatgttgctcattgtcatggaattttgatcacatcttactcctacattcgattgatgcaggatgacattagcaggtatgactggcactatgtgatcttggacgaaggacacaaaattcgaaatccaaatgctgctgtcacccttgcttgcaaacagtttcgcacccctcatcggatcattctgtctggctcaccgatgcaaaataacctccgagagctgtggtcgctctttgacttcatcttcccgggaaagttaggcacgttgcctgtgtttatggagcagttctccgtccccatcaccatggggggatattcaaatgcttccccagtacaggtcaaaactgcttacaagtgtgcatgtgtcttacgagataccataaatccatacctactgcggagaatgaagtcagatgtcaagatgagcctttctttgccagataaaaatgaacaggtcttattttgccgtcttacagatgagcagcataaagtctaccaaaatttcgttgattccaaagaagtttacaggattctcaatggagagatgcagattttctccggacttatagccctaagaaaaatttgcaaccaccctgatctcttttctggaggtcccaagaatctcaaaggtcttcctgatgatgaactagaagaagatcagtttgggtactggaaacgttctgggaaaatgattgttgttgagtctttgttgaaaatatggcacaagcagggtcagcgagtattgctgttttctcagtcaaggcagatgctggacatacttgaagtattccttagagcccaaaagtatacctatctcaagatggatggtaccactacaatagcttcaagacagccactgattacgagatacaatgaggacacatccatatttgtgtttcttctgaccacgcgggtgggcggcttaggtgtcaacctgacgggggcaaacagagttgtcatctatgacccagactggaacccaagcacggacacgcaggcccgggagcgagcatggagaataggccagaagaagcaagtgactgtgtacaggctcctgactgcgggcaccattgaagaaaagatctaccaccgacaaatcttcaagcagtttttgacaaatagagtgctaaaagacccaaaacaaaggcggtttttcaaatccaatgatctctatgagctatttactctgactagtcctgatgcatcccagagcactgaaacaagtgcaatttttgcaggaactggatcagatgttcagacacccaaatgccatctaaaaagaaggattcaaccagcctttggagcagaccatgatgttccaaaacgcaagaagttccctgcttctaacatatctgtaaatgatgccacatcatctgaagagaaatctgaggctaaaggagctgaagtaaatgcagtaacttctaatcgaagtgatcctttgaaagatgaccctcacatgagtagtaatgtaactagcaatgataggcttggagaagagacaaatgcagtatctggaccagaagagttgtcagtgattagtggaaatggggaatgttcaaattcttcaggaacaggcaaaacttctatgccatctggtgatgaaagcattgatgaaaagttaggtctttcttacaaaagagaaagacccagccaggctcaaacagaagctttttgggagaataaacaaatggaaaataatttttataagcacaagtcaaaaacaaaacatcatagtgtggcagaagaagagaccctggagaaacatctgagaccaaagcaaaagcctaagaactctaagcattgcagagacgccaagtttgaaggaactcgaattccacacctggtgaagaaaaggcgttaccagaagcaagacagtgaaaacaagagtgaggccaaggaacagagcaatgacgattatgttttggaaaagcttttcaaaaaatcagttggcgtgcacagtgtcatgaagcacgatgccatcatggatggagccagcccagattatgtactggtggaggcagaagccaaccgagtggcccaggatgccctgaaagcactgaggctctctcgtcagcggtgtctgggagcagtgtctggtgttcccacctggactggccacagggggatttctggtgcaccagcaggaaaaaagagtagatttggtaagaaaaggaattctaacttctctgtgcagcatccttcatcaacatctccaacagagaagtgccaggatggcatcatgaaaaaggagggaaaagataatgtccctgagcattttagtggaagagcagaagatgcagactcttcatccgggcccctcgcttcctcctcactcttggctaaaatgagagctagaaaccacctgattctgccagagcgtttagaaagtgaaagcgggcacctgcaggaagcttctgccctgctgcccaccacagaacacgatgaccttctggtggagatgagaaacttcatcgctttccaggcccacactgatggccaggccagcaccagggagatactgcaggagtttgaatccaagttatctgcatcacagtcttgtgtcttccgagaactattgagaaatctgtgcactttccatagaacttctggtggtgaaggaatttggaaactcaagccagaatactgctaaacaacattgcttcctaaactttcaagtccctttttctaacgggcatttctgattattaatttattattaataatcatgtttgtcaatggaagttggctgcacttgatgtttgtttgcatgatgtctacctcagaattaaaactttaaggaagg

SEQ ID NO: 3 - DNA, 3660 bp, Homo Sapiens
  ref <308>: DATABASE ACCESSION NUMBER: Genbank / D16305.1
  ref <309>: DATABASE ENTRY DATE: 2008-09-26
  ref <313>: RELEVANT RESIDUES IN SEQ ID NO: (1)..(3660)
cacccccttcatgggggtccagggcctctggaagctgctggagtgctccgggcggcaggtcagccccgaagcgctggaagggaagatcctggctgttgatattagcatttggttaaaccaagcacttaaaggagtccgggatcgccatgggaactcaatagaaaatcctcatcctctcactttgtttcatcggctctgcaaactcttattttttcgaattcgtcctatttttgtgtttgatggggatgctccactattgaagaaacagactttggtgaagagaaggcagagaaaggacttagcgtccagtgactccaggaaaacgacagagaagcttctgaaaacatttttgaaaagacaagccatccaaacttccttcagaagccaaagagatgaagcactacccagtcttacccaagttcgaagagaaaacgacctctatgttttgcctcctttacaagaggaagaaaaacacagttcagaagaggaagatgaaaaagaatggcaagaaagaatgaatcaaaaacaagcattacaggaagagttctttcataatcctcaagcgatagatattgagtctgaggacttcagcagcctgccccctgaagtaaagcatgaaatcttgactgatatgaaagagttcaccaagcgcagaagaacattatttgaagcaatgccagaggagtctgatgacttttcacagtaccaactcaaaggcttgcttaaaaagaactatctgaaccagcatatagaacatgtccaaaaggaagtgaatcagcaacattcaggacacatccgaagcagtcatgaagatgaagggggctttctgaaggaggtagagtcaaggagagtggtctctgaagacacttcacattacatcttgataaaaggtattcaagctaagacagttgcagaagtggattcagagtctcttccttcttccagcaaaatgcacggcatgtcttttgacgtgaagtcatctccatgtgaaaaactgaagacagagaaagagcctgatgctacccctccttctccaagaactttactagctatgcaagctgccctgctgggaagtagctcagaagaggagctggagagtgaaaatcgaaggcaggcccgtgggaggaacgcacctgctgctgtagacgaaggctccatatcaccccggactctttcagccattaagagagctcttgacgatgacgaagatgtaaaagtgtgtgctggggatgatgtgcagacgggagggccaggagcagaagaaatgcgtataaacagctccaccgagaacagtgatgaaggacttaaagtgagagatggaaaaggaataccgtttactgcaacacttgcgtcatctagtgtgaactctgcagaggagcacgtagccagcactaatgaggggagagagcccacagactcagttccaaaagaacaaatgtcacttgttcacgtggggactgaagcctttccgataagtgatgagtctatgattaaggacagaaaagatcggctgcctctggagagtgcagtggttagacatagtgacgcacctgggctcccgaatggaagggaactgacaccggcatctccaacttgtacaaattctgtgtcaaagaatgaaacacatgctgaagtgcttgagcagcagaacgaactttgcccatatgagagtaaattcgattcttctcttctttcaagtgatgatgaaacaaaatgtaaaccgaattctgcttctgaagtcattggccctgtcagtttgcaagaaacaagtagcatagtaagtgtcccttcagaggcagtagataatgtggaaaatgtggtgtcatttaatgctaaagagcatgagaattttctggaaaccatccaagaacagcagaccactgaatctgcaggccaggatttaatttccattccaaaggccgtggaaccaatggaaattgactcggaagaaagtgaatctgatggaagtttcattgaagtgcaaagtgtgattagtgatgaggaacttcaagcagaattccctgaaacttccaaacctccctcagaacaaggcgaagaggaactggtaggaactagggagggagaagcccctgctgagtccgagagcctcctgagggacaactctgagagggacgacgtggatggtgagccacaggaagctgagaaagatgcggaagattcgctccatgaatggcaagatattaatttggaggagttggaaactctggagagcaacctcttagcacagcagaattcactgaaagctcaaaaacagcagcaagaacggttcgctgctactgtcaccggacagatgttcctggaaagccaggaactcctgcgcctgttcggcattccctacatccaggctcccatggaagcagaggcgcagtgcgcagtcctggacctgactgatcagacttccggaaccatcactgatgacagtgatatctggctgtttggagcgcggcatgtctatagaaacttttttaataaaaacaagtttgtagaatattatcaatatgtggactttcacaatcaattgggattggaccggaataagttaataaatttggcttatttgcttggaagtgattataccgggaataccaactgtgggttgtgtaccgccatggaaattctcaatgaattccctgggcatggcctggaacctctcctaaaattctcagaatggtggcatgaagctcaaaaaaatccaaagataagacctaatcctcatgacaccaaagtgaaaaaaaaattacggacattgcaactcacccctggctttcctaacccagctgttgccgaggcctacctcaaacccgtggtggatgactcgaagggatcctttctgtgggggaaacctgatctcgacaaaattagcgaattttgtcagcggtatttcggctggaacagaacgaagacagatgaatctctgtttcctgtattaaagcaactcgatgcccagcagacacagctccgaattgattccttctttagattagcacaacaggagaaagaagatgctaaacgtattaagagccagagactaaacagagctgtgacatgtatgctaaggaaagagaaagaagcagcagccagcgaaatagaagcagtttctgttgccatggagaaagaatttgagctacttgataaggcaaaacgaaaaacccagaagagaggcataacaaataccttagaagagtcatcaagcctgaaaagaaagaggctttcagattctaaacgaaagaatacatgcggtggatttttgggggagacctgcctctcagaatcatctgatggatcttcaagtgaagatgctgaaagttcatctttaatgaatgtacaaaggagaacagctgcgaaagagccaaaaaccagtgcttcagattcgcagaactcagtgaaggaagctcccgtgaagaatggaggtgcgaccaccagcagctctagtgatagtgatgacgatggagggaaagagaagatggtcctcgtgaccgccagatctgtgtttgggaagaaaagaaggaaactaagacgtgcgaggggaagaaaaaggaaaacctaattaaaaaatatgtatcctctataattagttatgacagccatttgtaatgaatttgtgtgaaagacgtaataaaattaactggtagcacggtc

SEQ ID NO: 4 - DNA, 6 bp, Artificial Sequence
  Synthetic Primer
aataaa

SEQ ID NO: 5 - DNA, 30 bp, Artificial Sequence
  Synthetic Polynucleotide
acaacattgcttcctaaactttcaagtccc

SEQ ID NO: 6 - DNA, 106 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtgg

SEQ ID NO: 7 - DNA, 581 bp, Artificial Sequence
  Synthetic Polynucleotide
ttaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcaga

SEQ ID NO: 8 - DNA, 1974 bp, Artificial Sequence
  Synthetic Polynucleotide
atgctgggctttctgagcgccagacagaccggcctggaagatcccctgagactgagaagggccgagagcaccagaagagtgctgggcctggaactgaacaaggaccgggacgtggaacggatccacggcggaggcatcaacaccctggacatcgagcctgtggaaggccggtacatgctgagcggcggatccgatggcgtgatcgtgctgtacgacctggaaaacagcagccggcagagctactacacatgcaaggccgtgtgcagcatcggcagggaccaccccgatgtgcaccggtacagcgtggaaaccgtgcagtggtatccccacgacaccggcatgttcaccagcagcagcttcgacaagaccctgaaagtgtgggacaccaataccctgcagaccgccgacgtgttcaacttcgaggaaacagtgtacagccaccacatgagccccgtgtccaccaagcactgtctggtggccgtgggcaccagaggccctaaggtgcagctgtgcgatctgaagtccggcagctgcagccacatcctgcagggccacagacaggaaatcctggccgtgtcctggtcccccagatacgactacatcctggccaccgccagcgccgacagcagagtgaaactgtgggatgtgcggagagccagcggctgcctgatcaccctggatcagcacaacggcaagaaaagccaggccgtggaaagcgccaacaccgcccacaatggcaaagtgaacggcctgtgcttcaccagcgacggcctgcatctgctgacagtgggcaccgacaaccggatgcggctgtggaacagctccaacggcgagaataccctcgtgaactacggcaaagtgtgcaacaacagcaagaagggcctgaagttcaccgtgtcttgcggctgcagcagcgagttcgtgttcgtgccctacggcagcacaatcgccgtgtacaccgtgtactccggcgagcagatcacaatgctgaagggccactacaagaccgtggactgctgcgtgttccagagcaacttccaggaactgtacagcggcagccgggactgcaatatcctggcctgggtgcccagcctgtacgagcccgtgcctgacgacgatgagacaaccaccaagagccagctgaaccccgccttcgaggatgcctggtccagctccgatgaggaaggctgaatatcatctttagtacctttttgtctctgctgaaactttttaaatgagactgtgtttttttcaactgtatggtctattcctgacagctaaattagccctaaatgtgggtaatatttttcctcatgttttaaaatgaggttaatatttgcataaaatcctaaaacagacttctgtatagtttatttagtcaaaatgtgttccttgatcccagatgttgtggcctgggaaagccctcattgctacagtacaagtaacacaagtcgttgtacctcagttgtgaccttcagcagattttatgaactataagatgcagtctcagaggatcagcaagtggaggccatcagtattgactttctcttacttgctgtactatcagcctgctcgtttccacctttaagaatgattttgccaagaatgattatatcaaaaatagtagttgaaatggtaacatcaaaattattttattctttcttcttcatgtattcacatttttcagtggtttcatttaattaaccatgctttatgttaaacattttggggctcaatgtctcctactatccaaaatgtgcatcacaggaggcttttaactttgtgaaaatcccatgtttgctttattttattttaatgtcagaaggcagtttgcgctaatgcttgaactctttttctgtgaaactcattaaggtatgaccaaatcctgcctcattaattcaagcagaaaatatcctggcagggaatctggcttaaacatgaaatgctgtaataaaatttctatgttattgtctc

SEQ ID NO: 9 - DNA, 145 bp, Artificial Sequence
  Synthetic Polynucelotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 10 - DNA, 147 bp, Artificial Sequence
  Synthetic Polynucleotide
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 11 - DNA, 595 bp, Artificial Seuqence
  Synthetic Polynucleotide
attcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatc

SEQ ID NO: 12 - DNA, 3696 bp, Artificial Sequence
  Synthetic Polynucleotide
atgttgcctttacttctaggcctgtacggaagtgttacttctgctctaaaagctgcggaattgtacccgcggccgccgatgggagtacagggactctggaaactgctggaatgcagtgggagacaagtgagtccggaagcactggaaggtaagattctggctgtcgatatctctatctggctcaatcaggccctgaaaggggtcagggatagacatggtaactccatcgaaaatccccacccgcttaccctttttcaccgcctgtgcaagcttctgttcttcagaatcagaccaatcttcgtgtttgacggtgacgcacccctgctgaagaagcaaacactcgtaaaaaggcgacaacggaaggatctggcgagctctgactcccggaagaccacagagaagttgctcaagacattccttaagaggcaggccattcaaactagtttccgctcccaacgcgatgaagccctgccttctttgacacaagtgagaagagagaatgacctgtacgtcctgccaccgctccaggaggaagagaaacattcttcagaggaggaagatgaaaaggagtggcaggagcgaatgaatcagaaacaagccctgcaggaagaattttttcacaacccccaggccatcgacatcgaatccgaggactttagctccctcccgcccgaagtcaaacacgaaatcctcacagacatgaaagaatttaccaagcgccgaagaaccttgtttgaagccatgccagaagagtccgatgatttcagccaatatcaacttaagggactcttgaaaaagaattatttgaaccagcatatagagcatgttcagaaggaggtcaatcagcagcacagcggacatatccgatccagccatgaagatgaaggcgggttccttaaggaagtggaatcccgaagggtcgtatcagaagacacttctcattacatactgatcaaaggtattcaggcaaaaacggtcgcagaagtggattccgaaagtctcccaagcagctcaaaaatgcatggaatgtcattcgatgtgaagtcaagtccatgtgagaaactgaaaacagagaaagaaccagatgctacaccccccagccctcgaactctcttggcgatgcaggcggccctccttggttcctcaagcgaagaggaattggagtcagagaaccggagacaggcacgcggtcggaacgcacccgcagccgttgacgaaggatccattagcccccgcacccttagcgccatcaaacgggccctcgatgacgacgaggacgtgaaagtatgtgctggagacgacgttcagactggagggccgggggccgaagagatgagaattaacagcagcacagagaatagcgatgaaggcctcaaagtgagggatggtaaagggattccatttacagcgactctggcgtccagcagcgtgaattccgcagaagagcatgtcgcatcaacaaatgaaggccgcgaacccacagacagtgtgccgaaggagcaaatgtcacttgtccacgtcggaactgaagcctttccaatcagcgacgaatcaatgataaaagatcgcaaagacaggctgcccctcgaatccgccgtagtgaggcactccgacgctccggggcttcccaatggtagagagcttactccagcaagcccaacctgtactaattcagtttctaaaaacgagactcatgcagaagtgctcgagcagcagaacgagctttgcccatatgaaagtaaatttgatagttcactcctttcttctgacgacgagacaaagtgtaagccaaattccgcatctgaagtgataggccctgtatccctccaggaaacctcaagcatagtgtccgttccatctgaagcggttgacaacgtagagaatgtggtttcattcaacgcaaaagaacacgagaacttcctcgagaccattcaggagcagcaaacaactgagtctgccggccaggatctgattagcataccaaaagccgtcgaaccaatggagatcgacagcgaggagtctgaatccgatgggagcttcatcgaagtgcagagcgtgattagcgacgaggaactgcaggcagaattcccggaaaccagcaaaccaccgagcgaacagggtgaggaggaacttgtcggtacgagggaaggtgaggctcctgccgagagtgagagcctgcttagggataatagcgagagggacgacgtcgatggagagccccaggaagccgagaaggacgctgaagattctctccacgaatggcaggacattaatctggaggagctggaaaccttggaaagcaatctgcttgcccaacaaaactcactcaaggcgcagaaacaacagcaggaacgctttgcagccaccgttaccggacagatgtttctcgaatctcaagagttgcttcgcctgttcggcatcccttacatccaagcacctatggaggctgaagcccaatgtgcagtgctggacctcacagatcagactagcgggacaatcacggatgattcagatatctggctctttggcgctagacacgtctaccgcaattttttcaacaagaataagttcgtcgagtattatcagtatgtggacttccataaccagctgggactcgaccgcaataaactgatcaatttggcgtaccttctggggagcgactacacagggaacaccaattgcggcctgtgcacagcaatggaaattttgaacgaattccccggccacggtctggagccgctcctgaaatttagcgagtggtggcacgaagctcagaagaatcctaagattagaccaaacccgcatgacaccaaggtcaaaaaaaagcttaggacactgcagctcacacccgggttcccgaatcccgcggttgcagaagcttacctcaagccagtcgttgacgattccaagggtagcttcttgtggggaaaaccggatcttgataaaatctccgagttctgtcagagatattttggttggaatagaactaagaccgacgagtctcttttccctgttctgaaacaactggatgcacagcagacacaactgagaatcgattcctttttccgcctggctcaacaggaaaaagaagacgccaagcgcataaagagccaacgcctgaaccgggcagtgacctgcatgctgagaaaagaaaaggaagcagcagcaagtgagattgaagccgtgagtgtcgctatggagaaagaatttgagctcctggataaggctaaaagaaaaacccaaaaaagagggattactaatacccttgaagagtcatctagcttgaagcgaaaaagattgtccgattccaagcgcaagaacacgtgcggaggatttctcggcgaaacgtgccttagcgaatcctccgacgggtcaagttcagaggacgccgaatcttcttctttgatgaatgtgcaaagaagaactgccgccaaagagcccaagacatctgcaagtgacagccagaacagcgttaaggaagctccagttaaaaatggcggcgccacaacatcaagctcttccgactccgacgacgatggtggaaaagaaaagatggtactcgtgacagctcggtctgtttttggtaagaaaagaagaaagcttcggagggctagaggtcggaaaagaaaaaccggggatccagacatgataagatacattgatgagtttggacaaaccacaactagaatgcagtga

SEQ ID NO: 13 - DNA, 146 bp, Artificial Sequence
  Synthetic Polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaac

SEQ ID NO: 14 - DNA, 4479 bp, Artificial Seuqence
  Synthetic Polynucleotide
atgcccaatgagggcatccctcacagcagccagacacaagagcaggactgcctgcagagccagcctgtgtccaacaacgaggaaatggccatcaagcaagagtctggcggcgacggcgaggtggaagagtacctgagctttagaagcgttggcgacggcctgagcacatctgctgtgggatgtgcttctgccgctcctagaagaggacctgctctgctgcacatcgaccggcatcagattcaggccgtggaaccttctgcacaggccctggaactgcaaggcctgggagtcgatgtgtacgaccaggacgttctggaacagggcgtgctgcaacaggtggacaatgccattcacgaggccagcagagcctctcagctggtggacgtggaaaaagaatatcggagcgtgctggacgacctgaccagctgtacaaccagcctgcggcagatcaacaagatcatcgagcagctgtctccccaggccgccaccagcagagacatcaacagaaagctggacagcgtgaagcgccagaagtacaacaaagagcagcagctgaagaagatcaccgccaagcagaaacatctgcaggccattctcggcggagccgaagtgaagatcgaactggatcacgccagcctggaagaggatgccgaacctggaccaagcagcctgggctctatgctgatgcccgtgcaagagacagcctgggaagaactgatccggaccggccagatgacccctttcggaacacagatccctcagaagcaagagaagaaaccccggaagatcatgctgaacgaggcctccggcttcgagaagtacctggccgatcaggccaagctgagcttcgagagaaagaagcagggctgcaacaagagagccgccagaaaagctcccgctcctgtgacacctcctgctccagtgcagaacaagaacaagcccaacaagaaagcccgggtgctgagcaagaaagaggaacgcctgaagaaacacatcaagaagctgcagaagcgggccctgcagtttcaaggcaaagtgggcctgcctaaggccagaaggccttgggaaagcgacatgaggcctgaggccgagggcgattctgagggcgaagagagcgagtacttccccaccgaggaagaagaagaggaagaggacgacgaggttgagggcgccgaagctgatcttagcggagatggcaccgactacgagctgaagcctctgcctaaaggcggcaagaggcagaaaaaggtgccagtgcaagaaatcgacgacgacttcttcccatcctccggcgaagaagccgaagccgcctctgttggagaaggcggcggaggcggaagaaaagtgggcagatacagagatgacggcgacgaggactactacaagcagcggctgagaaggtggaacaagctgagactgcaggacaaagagaagaggctgaagctggaagatgacagcgaggaatccgacgccgagttcgacgagggctttaaggtgcccggcttcctgtttaagaagctgttcaagtaccagcagaccggcgtgcggtggctgtgggaactgcattgtcaacaggcaggcggaatcctgggcgacgaaatgggactgggcaagacaatccagattatcgccttcctggccggcctgtcctacagcaagatcagaacccggggcagcaactacagattcgaaggactgggccccaccgtgatcgtgtgtcctacaacagtgatgcaccagtgggtcaaagaattccacacctggtggcctccattcagagtggccatcctgcacgagacaggcagctacacccacaagaaagaaaagctgatccgcgacgtggcccactgccacggcatcctgatcacaagctacagctacatccggctgatgcaggacgacatcagcagatacgactggcactacgtgatcctggacgagggccacaagattcggaaccccaatgccgctgtgaccctggcctgcaagcagttcagaacccctcaccggatcatcctgagcggcagccccatgcaaaacaacctgagagagctgtggtccctgttcgacttcatcttccctggcaagctgggcaccctgcctgtgttcatggaacagttcagcgtgcccatcaccatgggcggctacagcaatgcctctcctgtgcaagtgaaaaccgcctacaagtgcgcctgcgtgctgcgggataccatcaatccttacctgctgcggcggatgaagtccgacgtgaagatgagcctgagcctgcctgacaagaacgaacaggtgctgttctgccggctgaccgacgagcagcacaaggtgtaccagaacttcgtggacagcaaagaggtgtaccggatcctgaacggcgagatgcagatcttcagcggactgatcgccctgcggaagatctgtaatcaccccgacctgttcagcggcggacccaagaatctgaagggcctgccagacgacgaactcgaggaagatcagtttggctactggaagcggagcggcaagatgatcgtggtggaaagcctgctgaagatttggcacaagcagggccagagagtgctgctgttcagccagagcagacagatgctggatatcctggaagtgttcctgcgggcccagaagtatacctacctgaagatggacggcaccaccacaatcgccagcagacagcctctgatcacccggtacaatgaggatacctccatcttcgtctttctgctgaccaccagagtcggcggactgggcgttaacctgactggcgccaatcgggtcgtgatctatgaccccgactggaaccccagcaccgacacacaggctagagagagagcttggagaatcggccagaaaaagcaagtgaccgtgtaccggctgctgaccgccggaacaatcgaagagaagatctaccaccggcagatctttaagcagttcctgaccaacagggtgctgaaggaccccaagcagagaagattcttcaagagcaacgacctgtatgagctgttcaccctgacaagccccgatgccagccagtctacagagacaagcgccatctttgccggcaccggctctgatgtgcagacccctaagtgtcacctgaagcggagaatccagcctgcctttggcgccgatcacgacgtgcccaagcggaagaagttccccgccagcaacatcagcgtgaacgatgccaccagctccgaggaaaagagcgaagccaaaggcgctgaagtgaacgccgtgaccagcaacagaagcgaccctctgaaggacgaccctcacatgagcagcaacgtgacctccaacgaccggctgggagaagagacaaatgccgtgtctggccccgaggaactgagcgtgatatctggcaatggcgagtgcagcaacagctctggcaccggcaagacctctatgccatctggcgacgagagcatcgacgagaagctgggcctgtcttacaagagagagcggccttctcaggcccagaccgaggccttttgggagaacaagcagatggaaaacaacttctacaagcacaagtccaagaccaagcaccacagcgtggccgaagaggaaaccctggaaaagcacctgaggcctaagcagaagcccaagaactccaagcactgccgggatgccaagttcgagggcaccagaattccccatctggtcaagaagcggcgctaccagaagcaggacagcgagaacaagtccgaggccaaagaacagtccaacgacgactacgtgctggaaaaactgttcaagaaaagcgtgggcgtgcacagcgtgatgaagcacgacgccattatggacggcgctagccccgattatgtgctggtggaagccgaggctaatagagtggcccaggatgccctgaaggccctgagactctccagacagagatgtctgggagctgtgtccggcgtgccaacatggacaggacacagaggaatttctggcgcccctgccggcaagaagtctcgctttggcaagaagagaaacagcaacttctccgtgcagcacccctccagcacaagccctacagagaagtgccaggacggcatcatgaagaaagaaggcaaggacaacgtccccgagcactttagcggcagagccgaggatgctgatagctctagtggacctctggccagcagttccctgctggctaagatgagagcccggaaccacctgatcctgccagagagactggaaagcgagtccggccatctgcaagaagctagcgcactgctgcctaccaccgagcacgatgatctgctggtcgagatgcggaacttcattgcctttcaagcccacacagacggccaggcctctaccagagagatcctgcaagagtttgagagcaagctgtccgcctctcagagctgcgtgttcagagagctgctgagaaacctgtgcaccttccacagaaccagcggaggcgaaggcatctggaagctgaaacccgagtactgc

SEQ ID NO: 15 - protein, 1185 aa, Homo Sapiens [sequence omitted]

SEQ ID NO: 16 - protein, 396 aa, Homo Sapiens
MLGFLSARQTGLEDPLRLRRAESTRRVLGLELNKDRDVERIHGGGINTLDIEPVEGRYMLSGGSDGVIVLYDLENSSRQSYYTCKAVCSIGRDHPDVHRYSVETVQWYPHDTGMFTSSSFDKTLKVWDTNTLQTADVFNFEETVYSHHMSPVSTKHCLVAVGTRGPKVQLCDLKSGSCSHILQGHRQEILAVSWSPRYDYILATASADSRVKLWDVRRASGCLITLDQHNGKKSQAVESANTAHNGKVNGLCFTSDGLHLLTVGTDNRMRLWNSSNGENTLVNYGKVCNNSKKGLKFTVSCGCSSEFVFVPYGSTIAVYTVYSGEQITMLKGHYKTVDCCVFQSNFQELYSGSRDCNILAWVPSLYEPVPDDDETTTKSQLNPAFEDAWSSSDEEG

SEQ ID NO: 17 - protein, 1493 aa, Homo Sapiens
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYNKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC